SEQUENCE LISTING (6 sequences)

SEQ ID NO: 1 - DNA, 20 bp, Homo sapiens
  source 1..20: mol_type = genomic DNA
gtcggactttgtgaaatacg

SEQ ID NO: 2 - DNA, 20 bp, Homo sapiens
  source 1..20: mol_type = genomic DNA
acgcgcacccacaagcggga

SEQ ID NO: 3 - DNA, 20 bp, Homo sapiens
  source 1..20: mol_type = genomic DNA
gtcggaggagatcatcacgc

SEQ ID NO: 4 - DNA, 20 bp, Homo sapiens
  source 1..20: mol_type = genomic DNA
ggcttcgaagacttcaccgg

SEQ ID NO: 5 - DNA, 20 bp, Homo sapiens
  source 1..20: mol_type = genomic DNA
ggtagtagcaaccaacggga

SEQ ID NO: 6 - DNA, 20 bp, Homo sapiens
  source 1..20: mol_type = genomic DNA
gtattactgatattggtggg

(19) United States
(12) Patent Application Publication
(10) Pub. No.: US 2025/0224398 A1
(43) Pub. Date: Jul. 10, 2025

(54) GASDERMIN E EXPRESSION IN HUMAN T CELLS AS A MARKER FOR PROINFLAMMATORY T CELL FUNCTIONS

(71) Applicant: LEIBNIZ-INSTITUT FÜR NATURSTOFF-FORSCHUNG UND INFEKTIONSBIOLOGIE E. V. HANS-KNÖLL-INSTITUT (HKI), Jena (DE)

(72) Inventor: Christina Zielinski, Jena (DE)

(21) Appl. No.: 18/852,977

(22) PCT Filed: Mar. 31, 2023
(86) PCT No.: PCT/EP2023/058531

§ 371 Date: Sep. 30, 2024

(30) Foreign Application Priority Data
Mar. 31, 2022 (LU) LU501764

(51) Int. Cl. G01N 33/569; G01N 33/50

(52) U.S. Cl. CPC G01N 33/56972; G01N 33/505; G01N 2333/545; G01N 2333/96466

(57) ABSTRACT

The present invention relates to a method, in particular an in vitro method, for diagnosing an inflammatory disease in a human patient, comprising detecting IL-1α producing Th17 cells in a sample comprising T cells obtained from said patient comprising detecting gasdermin E protein expression, wherein the presence of said IL-1α producing Th17 cells is indicative for an inflammatory disease in the human patient. The inflammatory disease is selected from the group of an inflammation that is caused or exacerbated by IL-1α producing Th17 cells, inflammation caused or related to danger signal IL-1α. The present invention further relates to methods for diagnosing the status of an inflammatory disease in a human patient, or for identifying an inflammation modulating compound, such as an anti-inflammatory compound. Furthermore, the present invention relates to a kit for performing the above methods. Finally, improved inflammation modulating, and in particular anti-inflammatory compounds or pharmaceutical compositions are provided.

The present invention relates to a method, in particular an in vitro method, for diagnosing an inflammatory disease in a human patient, comprising detecting IL-1α producing Th17 cells in a sample comprising T cells obtained from said patient comprising detecting gasdermin E protein expression, wherein the presence of said IL-1α producing Th17 cells is indicative for an inflammatory disease in the human patient. The inflammatory disease is selected from the group of an inflammation that is caused or exacerbated by IL-1α producing Th17 cells, inflammation caused or related to danger signal IL-1α. The present invention further relates to methods, in particular in vitro methods, for diagnosing the status of an inflammatory disease in a human patient, or for identifying an inflammation modulating compound, such as an anti-inflammatory compound. Furthermore, the present invention relates to a kit for performing the above methods as well as respective uses thereof. Finally, improved inflammation modulating, and in particular anti-inflammatory compounds or pharmaceutical compositions are provided.

### BACKGROUND OF THE INVENTION

The human immune system mounts pro-inflammatory immune responses as a prerequisite for efficient pathogen clearance. The responses are tailored to the invading microbial antigen and tissue microenvironment, resulting in very diverse context-specific inflammatory signatures (1).

T helper cells represent important executioners of antigen specific effector responses through their secretion of distinct cytokines. Th17 cells, in particular, are recognized for their anti-fungal functions through secretion of their signature cytokine IL-17A, which is regulated by the transcription factor ROR-γt.

Th17 cells also act as the main culprits in the pathogenesis of autoimmune diseases (2). It has previously been recognized that Th17 cells display functional heterogeneity (3). Pro- and anti-inflammatory functions are exerted by the differential co-expression of IL-17 with either IFN-γ or IL-10, respectively (4-7). Overall, this has shaped the concept of a Th17 cell dualism and has stirred a quest for signals and molecular targets that shift the balance between both functional Th17 cell outcomes for therapeutic applications (4, 6, 8).

A deeper understanding of the identity and mechanistic basis that confers pathogenic versus immunoregulatory Th17 cell fates, remains elusive.

IL-1 cytokines, of which IL-1α and IL-1β, represent the most prominent members, exert profound inflammation upon binding to their shared IL-1R1 receptor, which is ubiquitously expressed (9). They induce rapid innate inflammation upon release from antigen presenting cells, but also orchestrate adaptive immunity by promoting Th17 cell polarization and T cell pathogenicity (4, 10). Unlike most other cytokines, they lack a signal peptide and are therefore secreted by an unconventional, endoplasmic reticulum-Golgi-independent mechanism.

Pro-IL-1β requires enzymatic cleavage before release into the extracellular space. The NLRP3 inflammasome is a multimeric cytosolic protein complex that assembles upon microbial infection and cellular damage and recruits caspase-1 for IL-1β cleavage (11). Recently it was demonstrated that IL-1β release also requires caspase 1 mediated gasdermin D cleavage and pore formation in a process called pyroptosis, an inflammatory form of cell death (12, 13). IL-1α, on the other hand, is thought to be processed independently of the NLRP3 inflammasome by yet poorly understood regulatory checkpoints (9). Despite these completely distinct pathways for the maturation and release of IL-1β and IL-1α, both cytokines are jointly produced by cells of the innate immune system, suggesting yet to be identified coregulatory routes.

U.S. Pat. No. 11,208,399 B2 discloses pyridazin-3-yl phenol compounds that inhibit NOD-like receptor protein 3 (NLRP3) inflammasome activity. The invention further relates to the processes for their preparation, pharmaceutical compositions and medicaments containing them, and their use in the treatment of diseases and disorders mediated by NLRP3.

Tsuchiya K. (in: Switching from Apoptosis to Pyroptosis: Gasdermin-Elicited Inflammation and Antitumor Immunity. Int J Mol Sci. 2021; 22(1): 426. Published 2021 Jan. 4. doi: 10.3390/ijms22010426) discloses that pyroptosis is a necrotic form of regulated cell death. Gasdermines (GSDMs) are a family of intracellular proteins that execute pyroptosis. While GSDMs are expressed as inactive forms, certain proteases proteolytically activate them. The N-terminal fragments of GSDMs form pores in the plasma membrane, leading to osmotic cell lysis. Pyroptotic cells release pro-inflammatory molecules into the extracellular milieu, thereby eliciting inflammation and immune responses. Recent studies have significantly advanced our knowledge of the mechanisms and physiological roles of pyroptosis. GSDMs are activated by caspases and granzymes, most of which can also induce apoptosis in different situations, for example where the expression of GSDMs is too low to cause pyroptosis; that is, caspase/granzyme-induced apoptosis can be switched to pyroptosis by the expression of GSDMs. Pyroptosis appears to facilitate the killing of tumor cells by cytotoxic lymphocytes, and it may also reprogram the tumor microenvironment to an immunostimulatory state. Understanding pyroptosis may help the development of cancer immunotherapy.

WO 2019/180450A1 discloses that pyroptosis is a novel biomarker and target for therapy in liver failure such as acute liver failure (ALF) and acute-on-chronic liver failure (ACLF). Gasdermin D (GSDMD), caspase 4, caspase 5, or Interleukin 1 alpha (IL-1α) can be detected and quantified in serum or plasma, and used as biomarkers for outcome in liver failure such as acute liver failure (ALF) and ACLF and other diseases involving aberrant pyroptosis. By antagonising GSDMD, caspase 4, caspase or Interleukin 1 alpha (IL-1α) many of the unwanted consequences or symptoms of liver failure such as acute liver failure (ALF) and acute-on-chronic liver failure (ACLF) may be reduced.

Wang Y, et al. (in: GSDME mediates caspase-3-dependent pyroptosis in gastric cancer. Biochem Biophys Res Commun. 2018 Jan. 1; 495(1): 1418-1425. doi: 10.1016/j.bbrc.2017.11.156. Epub 2017 Nov. 26. PMID: 29183726) disclose that the mechanism of the chemotherapy drugs on gastric cancer is not completely understood. Pyroptosis is a form of programmed cell death and plays a critical role in immunity. The role of pyroptosis on cancer cells is less known. In the study, they treated SGC-7901 and MKN-45 with 5-FU and found that the cell viability was significantly decreased. The release of LDH and the percentage of PI and APC Annexin-V double positive cells after 5-FU treatment were elevated compared to control group. Moreover, there were large bubbles blowing from the membrane of 5-FU-treated cells and the cleavage of GSDME but not GSDMD, which were blocked by the silence or specific inhibitor of caspase-3. Additionally, GSDME knockout by CRISPR-Cas9 switched 5-FU induced pyroptosis into apoptosis in SGC-7901. In conclusion, they find that GSDME switches chemotherapy drug-induced caspase-3 dependent apoptosis into pyroptosis in gastric cancer cells.

WO 2021/143455A1 discloses the diagnostic use of the gasdermin E-mediated pyroptosis pathway in the prediction and/or treatment of cytokine release syndrome, the use of a reagent for specifically detecting the activity or level of gasdermin E protein or gene in the preparation of a kit for predicting the risk of cytokine release syndrome occurring in a subject, and the use of a reagent for blocking and/or inhibiting the activity or level of gasdermin E protein or gene in the preparation of a drug for inhibiting and/or reducing the occurrence of cytokine release syndrome in a subject. Specifically, the use is to overcome the adverse consequences of cytokine release syndrome caused by CAR T in the treatment of tumors in the prior art, new strategies are needed to control the cytokine release syndrome, especially cytokines, while maintaining or improving the efficacy of CAR T cell therapy. Th17 cells are not mentioned.

It is clear from the above that there is a need in the art to provide methods that identify and diagnose the status of Th17 cells in the context of inflammatory diseases. Furthermore, new and effective treatments of conditions using compounds that selectively inhibit or reduce the pro-inflammatory activity of Th17 cells. It is therefore an object of the present invention, to provide such assays and methods. Other objects and advantages of the present invention will become apparent upon studying the following description of the invention.

In a first aspect thereof, the present invention solves the above problem by providing a method for diagnosing an inflammatory disease in a human patient, comprising detecting IL-1α producing Th17 cells in a sample comprising T cells obtained from said patient comprising detecting gasdermin E protein expression, wherein the presence of said IL-1α producing Th17 cells is indicative for an inflammatory disease in the human patient. Preferably, the IL-1α as produced by the Th17 cells and/or as optionally detected as well is secreted. Further preferred is the method according to the present invention, wherein the detection of the gasdermin E protein expression comprises detection of gasdermin E protein pore formation.

In general, the method according to the present invention can be used to diagnose any inflammatory disease that is caused or exacerbated by IL-1α producing Th17 cells, such as an inflammation caused or related to danger signal IL-1α. Further preferred examples are selected from the group of autoinflammatory Schnitzler syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout.

Further preferred is the method according to the present invention, further comprising the step of detecting the relative amount of the IL-1α producing Th17 cells per volume of the sample and/or per overall Th17 cell population in said sample. The method according to the present invention may further comprise comparing the relative amount of the IL-1α producing Th17 cells as detected to a control sample and/or an earlier sample taken from the same patient.

In a second aspect thereof, the present invention solves the above problem by providing a method for diagnosing the status and/or status of an inflammatory disease in a human patient, comprising performing the method according to the present invention as above, and diagnosing an exacerbated state of the inflammatory disease if an increase of the relative amount of the IL-1α producing Th17 cells is detected or a reduced state of the inflammatory disease if an decrease of the relative amount of the IL-1α producing Th17 cells is detected.

In a third aspect thereof, the present invention solves the above problem by providing a method for identifying an inflammation modulating compound, comprising the steps of: a) contacting at least one inflammation modulating candidate compound with the pore forming part of human gasdermin E protein (GSDME-N), and b) detecting the inhibition or increase of assembly/pore formation of GSDME-N in the presence of said candidate compound, when compared to the absence of said candidate compound, wherein the inhibition or increase of assembly/pore formation of GSDME-N identifies an inflammation modulating compound. Preferably, this aspect of the method according to the present invention is performed in vitro, e.g. using liposomes or other extracellular systems, or in a recombinant cell, such as, for example, a human Th17 cell, optionally lacking the gasdermin E gene.

In a fourth aspect thereof, the present invention solves the above problem by providing a method for identifying an inflammation modulating compound, comprising the steps of: a) contacting at least one inflammation modulating candidate compound with a cell expressing human gasdermin E protein, b) inducing gasdermin E expression in said cell, and c) detecting the inhibition or increase of assembly/pore formation of GSDME in the presence of said candidate compound, when compared to the absence of said candidate compound, wherein the inhibition or increase of assembly/pore formation of GSDME identifies an inflammation modulating compound. Preferably, the cell as used is a human Th17 cell. Preferably, the modulation is inhibition of assembly/pore formation of GSDME and the compound is an anti-inflammatory compound.

In general, the candidate compound may be selected from any suitable compound that can be used to treat or present any inflammatory disease that is caused or exacerbated by IL-1α producing Th17 cells, such as an inflammation caused or related to danger signal IL-1α. Further preferred examples are selected from the group of autoinflammatory Schnitzler syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout. The candidate compound may be selected from the group consisting of a chemical molecule, a molecule selected from a library of small organic molecules, a molecule selected from a combinatory library, a cell extract, in particular a plant cell extract, a small molecular drug, a protein, a protein fragment, a molecule selected from a peptide library, and an antibody or fragment thereof.

Preferably, this aspect of the method according to the present invention is performed in vivo or in vitro, in solution or comprises the candidate compound molecule bound or conjugated to a solid carrier.

In a fifth aspect thereof, the present invention solves the above problem by providing an inflammation modulating compound as identified according to a method according to the present invention, or a pharmaceutical composition comprising said inflammation modulating compound, together with a pharmaceutically acceptable carrier. Another aspect of the present invention relates to a method for producing a pharmaceutical composition comprising formulating at least one inflammation modulating compound as identified herein with a pharmaceutically acceptable carrier.

In a sixth aspect thereof, the present invention solves the above problem by providing a method for preventing or treating inflammation in a subject, wherein the inflammatory disease is selected from the group of an inflammation that is caused or exacerbated by IL-1α producing Th17 cells, inflammation caused or related to danger signal IL-1α; autoinflammatory Schnitzler syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout, comprising administering to said subject an effective amount of an anti-inflammatory compound or a pharmaceutical composition comprising said inflammation modulating compound according to the present invention. In a seventh aspect thereof, the present invention solves the above problem by providing an inflammation modulating compound or the pharmaceutical composition comprising the inflammation modulating compound according to the present invention for use in the prevention or treatment of inflammation in a subject as above.

In an eighth aspect thereof, the present invention solves the above problem by providing a method for monitoring an inflammation modulating treatment or prophylaxis in a subject in need thereof, comprising a) providing an inflammation modulating treatment or prophylaxis to said subject as described herein, comprising administering to said subject an inflammation modulating compound or pharmaceutical composition according to the present invention, b) detecting the amount of IL-1α producing Th17 cells in a biological sample obtained from said subject according to a method according to the present invention, and c) comparing the amount(s) as detected in step b) with the amount in an earlier sample taken from said subject, and/or a control sample.

In a ninth aspect thereof, the present invention solves the above problem by providing a method for predicting or prognosing the success of, progress of and/or sensitivity for an inflammation modulating treatment or prophylaxis in a subject, comprising providing an inflammation modulating treatment or prophylaxis to said subject, comprising administering to said subject an inflammation modulating compound or pharmaceutical composition according to the present invention, performing the method according to the present invention, and detecting the amount of IL-1α producing Th17 cells in a biological sample obtained from said subject according to a method according to the present invention, wherein a decrease of the amount of the IL-1α producing Th17 cells when compared to an earlier sample taken from said subject, and/or a control sample is indicative for the success of, progress of and/or sensitivity for the inflammation modulating treatment or prophylaxis in the subject. Preferred is the method according to the present invention, wherein the subject further receives a second additional inflammation modulating prophylaxis or therapy, such as anti-inflammatory prophylaxis or therapy.

In the context of the present invention, the inventors showed that a subset of human Th17 cells engages an NLRP3-dependent signaling cascade for membrane pore formation by gasdermin E for the release of pro-inflammatory IL-1α. The gasdermin E (GSDME/DFNA5) cleavage in its linker by caspase-3 liberates the GSDME-N domain, which in non-Th17 cells mediates pyroptosis by forming pores in the plasma membrane. The inventors were able to exclude this type of cell death mechanism in Th17 cells, i.e., identifying this as cell type specific difference (see also below).

The so far overlooked population of IL-1α producing cells within the human Th17 cell subset displays enhanced features of pathogenicity compared to other Th17 cells. This finding was surprising because IL-1α production has previously been excluded as a T cell property (14).

Therefore, in a first aspect thereof, the present invention relates to a method for diagnosing an inflammatory disease in a human subject or patient, comprising detecting IL-1α producing Th17 cells in a sample comprising T cells obtained from said patient or subject comprising detecting gasdermin E protein expression, wherein the presence of said IL-1α producing Th17 cells is indicative for an inflammatory disease in the human patient.

In the context of the present invention, any suitable method to detect gasdermin E protein expression in said Th17 cells can be used. Expression can be detected directly, i.e. by identifying the production of gasdermin E-encoding mRNAs, e.g. using chip analysis, single-cell RNA sequencing, or the like. Another direct detection comprises detecting the production of the gasdermin E protein product in the cell, such as, for example, the full-length protein or the pore forming N-terminal part of said gasdermin E protein. Detection can be achieved with any suitable method to detect gasdermin E protein, such as antibodies, and the like. Preferred is the method according to the present invention, wherein the detection of the gasdermin E protein expression comprises detection of gasdermin E protein pore formation, again either by using antibodies density gradient centrifugation, analysis of membrane fractions, and optical methods to identify pores on the surface of cells.

Expression can also be detected indirectly, i.e. by identifying the production of other proteins or markers that are required in order to produce gasdermin pores, such as, for example, detecting the expression of at least one marker selected from NLRP3 inflammasome formation, calpain activity, caspase-3 activity, and caspase-8 activity in the Th17 cell as a marker for an IL-1α producing Th17 cell. Again, the detection can comprise marker-encoding mRNAs, e.g. using chip analysis, single-cell RNA sequencing, or the like. Another direct detection comprises detecting the production of the marker protein product in the cell. Detection can be achieved with any suitable method to detect the marker protein, such as antibodies, and the like. Preferred is the method according to the present invention, wherein the IL-1α as produced by the Th17 cells is detected, which is most convenient, since it is secreted, and not membrane bound (see below). Assays to detect IL-1α are known in the art.

The method according to the present invention is used to diagnose any inflammatory disease that is caused or exacerbated by IL-1α producing Th17 cells, such as an inflammation caused or related to danger signal IL-1α, because the population of IL-1α producing cells within the human Th17 cell subset displays enhanced features of pathogenicity compared to other Th17 cells. Further preferred examples are selected from the group of autoinflammatory Schnitzler syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout.

The biological sample or sample obtained from said patient or subject comprises Th17 cells. The sample may be a blood sample or a partially or fully homogeneous sample of Th17 cells as obtained from the patient or subject. For the purposes of the in vitro test and screenings as disclosed herein, the Th17 cells in the sample can be differentiated from naive CD4⁺ cells in the periphery in response to T cell receptor (TCR) antigen stimulation and activating cytokines secreted by antigen-presenting cells according to known protocols (Zielinski et al. Nature 2012, Braun JM & Zielinski CE Methods Mol Biol. 2014; 1193: 97-104. (Ivanov II, Mckenzie B S, Zhou L et al. (2006) The orphan nuclear receptor RORgammaT directs the differentiation program of proinflammatory IL-17+ T helper cells. Cell 126: 1121-1133). While differentiation was originally believed to be induced by IL-23, it was later demonstrated that Th17 development occurred independently of this cytokine. However, IL-23 is still thought to be important for Th17 maintenance and proliferation, and its receptor (IL-23R) is upregulated in activated Th17 cells (Ivanov II, Mckenzie B S, Zhou L et al. (2006) The orphan nuclear receptor RORgammaT directs the differentiation program of proinflammatory IL-17+ T helper cells. Cell 126: 1121-1133). The critical cytokine mediators of Th17 differentiation have instead been identified to be TGFβ in combination with IL-6 or IL-21 (Ivanov II, Mckenzie B S, Zhou L et al. (2006) The orphan nuclear receptor RORgammaT directs the differentiation program of proinflammatory IL-17+ T helper cells. Cell 126: 1121-1133, Dong C (2011) Genetic controls of Th17 cell differentiation and plasticity. Exp Mol Med 43: 1-6). IL-6 and IL-21 drive expression of Th17 transcriptional regulators via STAT3 signaling, committing CD4⁺ T cells to the Th17 lineage. Defects in this signaling pathway have been associated with decreased expression of IL-23R, key Th17-associated transcription factors, and effector cytokines such as IL-17A and IL-17F (Dong C (2011) Genetic controls of Th17 cell differentiation and plasticity. Exp Mol Med 43: 1-6). (This is a mix of mouse and human studies. The 2 added references focus on human Th17 cell differentiation).

Preferred is the method according to the present invention, further comprising the step of detecting the relative amount of the IL-1α producing Th17 cells per volume of the sample and/or per overall Th17 cell population in said sample. Preferred is the method according to the present invention, further comprising the step of comparing the relative amount of the IL-1α producing Th17 cells as detected to a control sample and/or an earlier sample taken from the same patient subject or patient. Suitable control samples may be taken from healthy volunteers and/or groups of donors, or may be differentiated Th17 cell preparations as above. Based on these embodiments, the method according to the present invention can be used to detect and analyze changes of the amount and/or relative population/amount of the Th17 cells over time, e.g. during the course of a treatment (see also below) and/or as an indicator of the status of the inflammatory disease in the patient or subject.

Therefore, another preferred aspect of the present invention relates to a method for diagnosing the status of an inflammatory disease in a human patient, comprising performing the method according to the present invention as disclosed above, and diagnosing an exacerbated state of the inflammatory disease, if an increase of the relative amount of the IL-1α producing Th17 cells is detected or a reduced state of the inflammatory disease if an decrease of the relative amount of the IL-1α producing Th17 cells is detected. As above, the method according to the present invention is used to diagnose any inflammatory disease that is caused or exacerbated by IL-1α producing Th17 cells, such as an inflammation caused or related to danger signal IL-1α, because the population of IL-1α producing cells within the human Th17 cell subset displays enhanced features of pathogenicity compared to other Th17 cells. Further preferred examples are selected from the group of autoinflammatory Schnitzler syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout.

In the context of the present invention, the patient or subject is preferably a mammal, such as a human.

Another preferred aspect of the present invention relates to a method for identifying an inflammation modulating compound, comprising the steps of: a) contacting at least one anti-inflammatory candidate compound with the pore forming part of human gasdermin E protein (GSDME-N), and b) detecting the inhibition or increase of assembly/pore formation of GSDME-N in the presence of said candidate compound, when compared to the absence of said candidate compound, wherein the inhibition or increase of assembly/pore formation of GSDME-N identifies an inflammation modulating compound.

Preferred is a method, wherein the modulation is inhibition of assembly/pore formation of GSDME and the compound is an anti-inflammatory compound.

This aspect of the present invention relates to a drug screening using the pore forming part of human gasdermin E protein (GSDME-N) and detecting the inhibition or increase of assembly/pore formation of GSDME-N in the presence of a candidate drug compound, when compared to the absence of said candidate compound. In general, any suitable method to detect gasdermin E protein pore formation or assembly of GSDME-N can be used, by using antibodies density gradient centrifugation, flow cytometry, western blot, analysis of membrane fractions, and optical methods to identify pores on the surface of cells. This method also includes the detection of pore assembly in vitro. This can be detected, for example using the liposome assay as disclosed by Xia (in: Monitoring gasdermin pore formation in vitro. Methods Enzymol. 2019; 625: 95-107. doi: 10.1016/bs.mic.2019.04.024. Epub 2019 May 23. PMID: 31455540; PMCID: PMC7533106, incorporated by reference). Since the structures of gasdermins depict well-conserved N-terminal and C-terminal domains which are linked through an intervening flexible hinge region which is a potential substrate site for proteases including caspases and granzymes, e.g. in the exemplary mouse GSDMA3 protein, GSDM-NT and GSDM-CT are joined through a long flexible linker (residues 234-263) which extends away from the main body making it accessible to activating enzymes, the method can be readily adjusted to gasdermin E protein pore formation. Other methods are known from the literature and to the person of skill. Preferred is the method according to the present invention, wherein the IL-1α as produced by the Th17 cells is detected, which is most convenient, since it is secreted, and not membrane bound (see below). Assays to detect IL-1α are known in the art.

Preferred is the method according to the present invention, wherein said method is performed in vitro or in a recombinant cell, such as, for example, a human Th17 cell, optionally lacking the autologous gasdermin E gene or having an inactivated autologous gasdermin E gene.

Another preferred aspect of the present invention relates to a method for identifying an inflammation modulating compound, comprising the steps of: a) contacting at least one inflammation modulating candidate compound with a cell expressing human gasdermin E protein, b) inducing gasdermin E expression in said cell, and c) detecting the inhibition or increase of assembly/pore formation of GSDME in the presence of said candidate compound, when compared to the absence of said candidate compound, wherein the inhibition or increase of assembly/pore formation of GSDME identifies an inflammation modulating compound. Preferred is a method, where the cell recombinantly expresses the pore forming part of human gasdermin E protein (GSDME-N).

Preferred is a method, wherein the modulation is inhibition of assembly/pore formation of GSDME and the compound is an anti-inflammatory compound.

This aspect of the present invention relates to a drug screening using the pore forming capacity of human gasdermin E protein (GSDME) and detecting the inhibition or increase of assembly/pore formation of GSDME-N in the presence of a candidate drug compound, when compared to the absence of said candidate compound in a cellular system. In general, any suitable method to detect gasdermin E protein pore formation or assembly of GSDME-N can be used, by using antibodies density gradient centrifugation, analysis of membrane fractions, and optical methods to identify pores on the surface of cells. This method also includes the indirect detection, i.e. by identifying the expression or production of other proteins or markers that are required in order to produce gasdermin pores, such as, for example, detecting the expression of at least one marker selected from NLRP3 inflammasome formation, calpain activity, caspase-3 activity, and caspase-8 activity in the Th17 cell as a marker for an IL-1α producing Th17 cell. Again, the detection can comprise marker-encoding mRNAs, e.g. using chip analysis, single-cell RNA sequencing, or the like. Another direct detection comprises detecting the production of the marker protein product in the cell. Detection can be achieved with any suitable method to detect the marker protein, such as antibodies, and the like. Preferred is the method according to the present invention, wherein the IL-1α as produced by the Th17 cells is detected, which is most convenient, since it is secreted, and not membrane bound (see below). Assays to detect IL-1α are known in the art.

Preferred is the method according to the present invention, wherein the step of inducing the gasdermin E expression in said cell comprises inducing NLRP3 inflammasome formation, calpain activity, caspase-3 activity, and/or caspase-8 activity. Further preferred is the method according to the present invention, wherein the inhibition or increase of assembly/pore formation of GSDME in the presence of said candidate compound comprises an inhibition or induction of the expression of gasdermin E and/or caspase-3 in said cell (i.e. the lack of cleaving the linker of the full length of gasdermin E protein), and/or a reduction or induction of the expression and/or secretion of IL-1α of said cell.

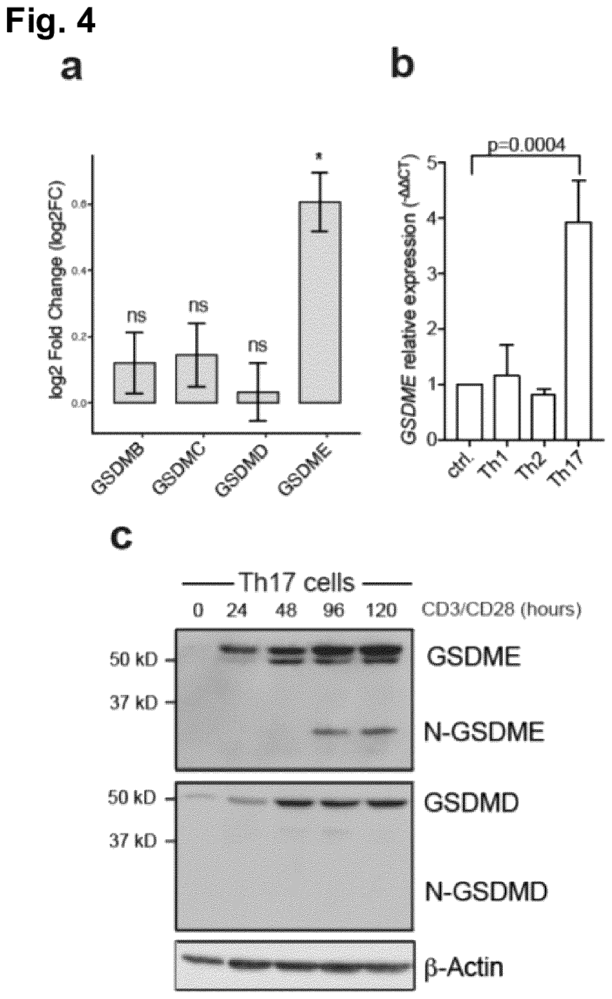

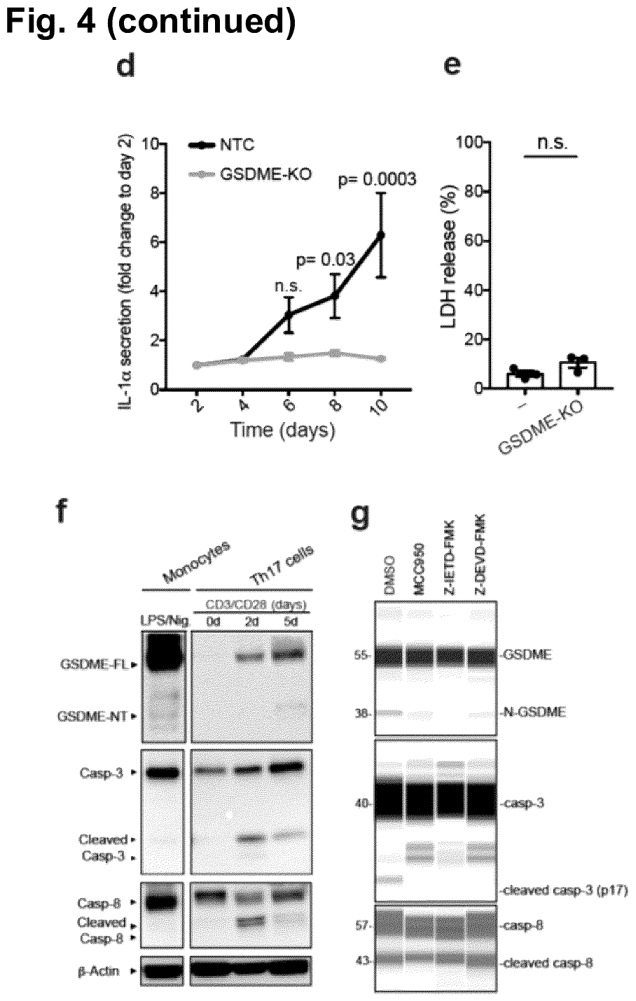

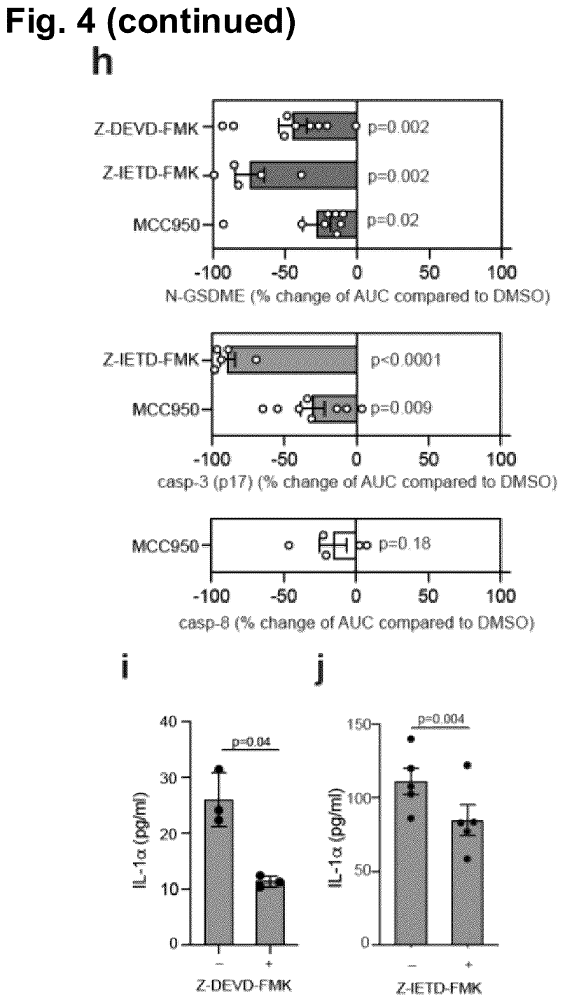

Some examples are known from the literature, and are disclosed herein, IL-1α secretion by Th17 cells was significantly inhibited by NLRP3 inflammasome inhibition with MCC950 (FIG. 4E), and Howley B (in: Caspases as therapeutic targets. J Cell Mol Med. 2008; 12 (5A): 1502-1516. doi: 10.1111/j.1582-4934.2008.00292.x) disclose other examples. Importantly, these data clearly established a critical role of the NLRP3 inflammasome for the secretion of IL-1α by human Th17 cells.

Further preferred is the method according to the present invention, wherein the cell as used is a human Th17 cell.

In the context of the present invention, the candidate compound can be selected from the group consisting of a chemical organic molecule, a molecule selected from a library of small organic molecules (molecular weight less than 500 Da), a molecule selected from a combinatory library, a cell extract, in particular a plant cell extract, a small molecular drug, a protein, a protein fragment, a molecule selected from a peptide library, an antibody or fragment thereof.

These candidate molecules may also be used as a basis to screen for improved compounds. Thus, preferred is the method according to the present invention, wherein after the identification of the anti-inflammatory compound, the method further comprises the step of chemically modifying the compound. In general, many methods of how to modify compounds of the present invention are known to the person of skill, and are disclosed in the literature.

Modifications of the compounds will usually fall into several categories, for example a) mutations/changes of amino acids into different amino acids, b) chemical modifications, e.g. through the addition of additional chemical groups, c) changes of the size/length of the compound, and/or d) the attachment of additional groups to the molecule (including marker groups, labels, linkers or carriers, such as chelators). In a next step, the modified compound is tested again in at least one of tests as above, and if the property of the compound is improved compared to its unaltered state. In the context of the present invention, an “improved” binding comprises both scenarios where the modified compound binds to the same extent as the unmodified (i.e. starting) compound, although the compound has been modified (e.g. by dimerization or by adding markers or other groups). Preferred is a compound as modified that exhibits a stronger binding to the target, e.g. gasdermin E. Also preferred is a compound that shows a longer binding to the target, or a binding fragment thereof, for example because of an improved stability of said modified compound in vitro or in vivo.

Assays to detect binding of the compound to the target are well known to the person of skill and preferably include mass spectrometry, NMR assays, pull-down assays, or the like.

Preferred is the method according to the present invention, wherein said contacting is in vivo or in vitro, in solution or comprises the candidate compound molecule bound or conjugated to a solid carrier. Respective formats are also described in the art, and known to the person of skill.

In another aspect of the invention, detecting the binding comprises a detecting competitive binding of the compound, for example in competition to a known inhibitor or an unaltered compound as identified.

Yet another aspect of the present invention then relates to an inflammation modulating compound as identified according to a method according to the present invention or a pharmaceutical composition comprising said inflammation modulating compound, together with a pharmaceutically acceptable carrier. Preferred is an anti-inflammatory compound.

Pharmaceutical compositions as used may optionally comprise a pharmaceutically acceptable carrier. Pharmaceutically acceptable carriers or excipients include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc.), humectants (e.g. propylene, glycol, glycerol, sorbitol). Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologic, 5^{th} Ed., Govi-Verlag Frankfurt (1997). The person skilled in the art knows suitable formulations for peptides and will readily be able to choose suitable pharmaceutically acceptable carriers or excipients, depending, e.g., on the formulation and administration route of the pharmaceutical composition.

The pharmaceutical composition can be administered orally, e.g. in the form of pills, tablets, coated tablets, sugar coated tablets, hard and soft gelatin capsules, solutions, syrups, emulsions or suspensions or as aerosol mixtures. Administration, however, can also be carried out rectally, e.g. in the form of suppositories, or parenterally, e.g. in the form of injections or infusions, or percutaneously, e.g. in the form of ointments, creams or tinctures.

In addition to the aforementioned compounds of the invention, the pharmaceutical composition can contain further customary, usually inert carrier materials or excipients. Thus, the pharmaceutical preparations can also contain additives, such as, for example, fillers, extenders, disintegrants, binders, glidants, wetting agents, stabilizers, emulsifiers, preservatives, sweetening agents, colorants, flavorings or aromatizers, buffer substances, and furthermore solvents or solubilizers or agents for achieving a depot effect, as well as salts for changing the osmotic pressure, coating agents or antioxidants. They can also contain the aforementioned salts of two or more compounds of the invention and also other therapeutically active substances as described herein.

Another aspect of the present invention relates to a method for producing a pharmaceutical composition comprising formulating at least one inflammation modulating compound as identified herein with a pharmaceutically acceptable carrier.

Yet another aspect of the present invention then relates to a method for preventing or treating inflammation in a subject, wherein the inflammatory disease is selected from the group of an inflammation that is caused or exacerbated by IL-1α producing Th17 cells, inflammation caused or related to danger signal IL-1α; autoinflammatory Schnitzler Syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout, comprising administering to said subject an effective amount of an inflammation modulating compound or a pharmaceutical composition comprising said inflammation modulating compound according to the present invention.

Preferred is the method according to the present invention, wherein the inflammation modulating treatment or prophylaxis in the patient or subject comprises administering an effective amount of the compound as identified herein, a pharmaceutical composition as described.

As mentioned herein, the compound is administered to said subject in an effective dosage. This dosage can vary within wide limits and is to be suited to the individual conditions in each individual case. For the above uses, the appropriate dosage will vary depending on the mode of administration, the particular condition to be treated and the effect desired. In general, however, satisfactory results are achieved at dosage rates are as above, e.g. of about 1 to 100 mg/kg animal body weight particularly 1 to 50 mg/kg. Suitable dosage rates for larger mammals, for example humans, are of the order of from about 10 mg to 3 g/day, conveniently administered once or in divided doses, e.g. 2 to 4 times a day, or in sustained release form. In general, a daily dose of approximately 10 mg to 100 mg, particularly 10 to 50 mg, per human individual is appropriate in the case of the oral administration. An effective concentration to be reached at the cellular level can be set at between 50 to 200 μM, preferably at about 100 μM. Particularly preferred is topical application, such as to the airways by inhalation. In these cases, the dosage can be conveniently reduced to between 0.1 to 10 mg/dose, preferably 0.2 to 5 mg per dose, which equals about 3 to about 80 μg per kilogram for a 70 kg subject.

It is to be understood that the present compound and/or a pharmaceutical composition comprising the present compound is for use to be administered to a human patient. The term “administering” means administration of a sole therapeutic agent or in combination with another therapeutic agent. It is thus envisaged that the pharmaceutical composition of the present invention are employed in co-therapy approaches, i.e. in co-administration with other medicaments or drugs and/or any other therapeutic agent which might be beneficial in the context of the methods of the present invention. Nevertheless, the other medicaments or drugs and/or any other therapeutic agent can be administered separately from the compound for use, if required, as long as they act in combination (i.e. directly and/or indirectly, preferably synergistically) with the present compound for use.

Thus, the compounds of the invention can be used alone or in combination with other active compounds-for example with medicaments already known for the treatment of the aforementioned diseases, whereby in the latter case a favorable additive, amplifying or preferably synergistically effect is noticed. Suitable amounts to be administered to humans range from 1 to 500 mg, in particular 5 mg to 100 mg, such as between 1 and 10 mg/kg/day oral dose. An effective concentration to be reached at the cellular level can be set at between 50 to 200 μM, preferably at about 100 μM.

In the medical use aspects of the present invention, the compound (for use) can be provided and/or is administered as a suitable pharmaceutical composition, such as a tablet, capsule, injection, granule, powder, sachet, reconstitutable powder, dry powder inhaler, inhalation, and/or chewable. Such solid formulations may comprise excipients and other ingredients in suitable amounts. Such solid formulations may contain e.g. cellulose, cellulose microcrystalline, polyvidone, in particular FB polyvidone, magnesium stearate and the like. Administration, however, can also be carried out rectally, e.g. in the form of suppositories, or parenterally, e.g. in the form of injections or infusions, or percutaneously, e.g. in the form of ointments, creams or tinctures.

The inflammation modulating compound or the pharmaceutical composition comprising the inflammation modulating compound according to the present invention is for use in the prevention or treatment of inflammation in a subject, wherein the inflammatory disease is selected from the group of an inflammation that is caused or exacerbated by IL-1α producing Th17 cells, inflammation caused or related to danger signal IL-1α; preferably autoinflammatory Schnitzler syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout.

An example for the treatment of a disease using the pro-inflammatory effects of gasdermin pore formation according to the present invention is the induction of phagocytosis for the treatment of C. albicans (see below).

Yet another aspect of the present invention then relates to a method, such as diagnostic method, for monitoring an inflammation modulating treatment or prophylaxis in a subject in need thereof, comprising a) providing an anti-inflammatory treatment or prophylaxis to said subject, comprising administering to said subject an inflammation modulating compound or pharmaceutical composition according to the present invention, b) detecting the amount of IL-1α producing Th17 cells in a biological sample obtained from said subject according to a method according to the present invention, and c) comparing the amount(s) as detected in step b) with the amount in an earlier sample taken from said subject, and/or a control sample. Preferred is the method, wherein decrease of the amount of IL-1α producing Th17 cells is indicative for the success of, progress of and/or sensitivity for the inflammation modulating treatment or prophylaxis in the mammalian subject. The method may further comprise adjusting the treatment or prophylaxis of said subject or patient based on said monitoring. The attending physician will be readily able to make and apply respective treatment decisions, also taking into account additional patient parameters, if required.

Similarly, yet another aspect of the present invention then relates to a method, such as a diagnostic method, for predicting or prognosing the success of, progress of and/or sensitivity for an inflammation modulating treatment or prophylaxis in a subject, comprising providing an inflammation modulating treatment or prophylaxis to said subject, comprising administering to said subject an inflammation modulating compound or pharmaceutical composition according to the present invention, performing the method according to the present invention, and detecting the amount of IL-1α producing Th17 cells in a biological sample obtained from said subject according to a method according to the present invention, wherein a decrease of the amount of the IL-1α producing Th17 cells when compared to an earlier sample taken from said subject, and/or a control sample is indicative for the success of, progress of and/or sensitivity for the inflammation modulating treatment or prophylaxis in the subject. The method may further comprise adjusting the treatment or prophylaxis of said subject or patient based on said monitoring. The attending physician will be readily able to make and apply respective treatment decisions, also taking into account additional patient parameters, if required.

In another aspect of the methods according to the present invention, the subject or patient further receives a second additional inflammation modulating prophylaxis or therapy. This may lead to additional or even synergistic efficiency of the anti-inflammatory prophylaxis or therapy.

Yet another aspect of the present invention then relates to a diagnostic kit comprising materials for performing a method according to the present invention in one or separate containers, optionally together with auxiliary agents and/or instructions for performing said method.

Preferred is a diagnostic kit according to the present invention comprising at least one of an anti-inflammatory candidate molecule, a recombinantly expressed human gasdermin E, in particular the N-terminal fragment capable of pore formation/assembly, preferably bound or conjugated to a solid carrier. The kit may further comprise specific antibodies binding to the components of the kit, dyes and other labels, as well buffers and matrices for performing the methods as above.

The kit may be used in the methods of the invention, i.e. for identifying an anti-inflammatory compound, for monitoring an anti-inflammatory treatment or prophylaxis in a patient or subject in need thereof, and/or for predicting or prognosing the success of, progress of and/or sensitivity for an anti-inflammatory treatment or prophylaxis in a patient or subject.

The assembly of an innate supramolecular cluster along a caspase cleavage signaling pipeline demonstrated how molecular bricks of innate immune signaling can moonlight for adaptive immunity and the enforcement of a pathogenic T cell cytokine memory. Th17 cells have emerged as the culprits of autoimmune pathogenesis (36). Functional heterogeneity has, however, been unraveled by the identification of pro-versus anti-inflammatory Th17 cell fates based on their differential coexpression of IFN-γ and IL-10, respectively (3, 4). Using single-cell RNA sequencing, the inventors have identified a so far overlooked population of IL-1α producing cells within the human Th17 cell subset. It displayed enhanced features of pathogenicity compared to other Th17 cells. This finding was surprising because IL-1α production has previously been excluded as a T cell property (14). Species-specific differences might apply, given absence of IL-1α production by murine T cells, which have so far served as negative controls for IL-1α producing cells in the scientific literature (14).

The inventors found that IL-1α expression was uniquely confined to the Th17 cell fate as evidenced by its co-expression with IL-17A, regulation by RORγt, induction by the Th17 priming cytokines IL-1β and TGF-β and its Th17-associated chemokine receptor expression profile. These findings are consistent with transcriptional binding sites of RORγt and RORα in IL-1α enhancer and promotor regions. The inventors also observed that Th17 priming cytokines increased NLRP3, GSDME and CAPN2 expression. Th17 polarization therefore not only promoted pro-IL-1α induction but also its processing and extracellular exodus. Interestingly, IL-1α production propagated the pro-inflammatory Th17 cytokine memory through continuous autocrine self-amplification but also suppression of IL-10 expression. This autocrine feedback loop would be consistent with the previously discovered, but until now mechanistically unresolved, continuous IL-10 blockade, which can be established by an initial IL-1α stimulus that is provided by antigen presenting cells during the early Th17 cell priming phase (4). These findings point to a new treatment rationale, by which IL-1α-, rather than IL-1β-neutralizing antibodies, might break this chronic pathogenic feedback loop at the Th17 effector differentiation stage.

A unique property that has previously been assigned to IL-1α is its simultaneous localization in the cytoplasm as well as plasma membrane (30). This is thought to spatially restrict its bioavailability in favor of contact dependent effector functions. Surface IL-1α was reported to only require NfKB activation without a need for the inflammasome and caspase-1, which would be consistent with the TCR activation, which the inventors have applied (27). The inventors found, however, that Th17 cells, in contrast to monocytes, do not display membrane-bound IL-1α. This prompted the conclusion that this membrane-localizing property does not apply universally to all IL-1α producing cells. Extracellular cleavage of membrane-bound IL-1α needs to be considered as a potential mechanism for this Th17 cell specific phenotype. However, the inventors ruled out this possibility with CRISPR-Cas9 mediated depletion of granzyme B, the only T cell associated extracellular candidate protease (22). Together, this implies a more systemic bioavailability of Th17 cell derived IL-1α in comparison to that of innate cell sources.

Previous reports have demonstrated roles for the NLRP3 inflammasome in human Th1 (37) and murine Th17 cells (38). This is the first study to show NLRP3 expression and inflammasome activity in Th17 cells from healthy human blood. Unlike innate cells, T cells are not specialized in innate danger sensing, which could trigger the assembly of NLRP3-inflammasome components. However, elevation of cytoplasmic calcium (Ca²⁺) has previously been shown to bypass innate danger signaling for NLRP3 inflammasome activation (14, 39). This is in line with the inventors finding that TCR activation, which is accompanied by calcium flux, was a requirement for IL-1α release by human Th17 cells. The inventors found pro-caspase-1 and GSDMD expression in human Th17 cells from healthy donors, however, no evidence for their NLRP3 inflammasome regulated cleavage nor for IL-1β production. This suggests that also conventional NLRP3-inflammasome signalling, as previously reported in response to complement activation (37), HIV infection (40) or potential other stimuli, might be operative in human Th17 cells and potentially translate into IL-1β release upon exposure to appropriate, however yet to be identified, stimuli.

Unexpectedly, the inventors found the NLRP3 inflammasome to be completely repurposed for the production of IL-1α in TCR activated Th17 cells. The inventors observed that Th17 cells engaged an alternative NLRP3 signalling cascade via engagement of caspase-8. This might have been facilitated by the absence of caspase-1 cleavage, as competitive caspase-1 versus caspase-8 inflammasome recruitment has been demonstrated before (32, 41). Previously, murine T cells have been reported to display activation of the NLRP3 inflammasome-ASC-caspase-8 axis upon TCR and ATP stimulation, fuelling into the establishment of a pathogenic Th17 cell phenotype. This was, however, exerted by IL-1β- and not IL-1α-release through a mechanism that remains to be elucidated (38). In human Th17 cells, instead, the inventors found IL-1α production to be dependent on caspase-8 cleavage. Inhibition of caspase-8 cleavage translated into blockage of the caspase-3-GSDME axis and thus in inhibition of the tunnelled IL-1α-exit. Cumulatively, this uncovered a signalling cascade was not yet observed in T cells.

An intriguing observation of the inventor's study was the identification of GSDME expression and its cleavage in T cells. Interestingly, it was selectively induced by TCR activation and promoted by Th17 cell polarizing conditions. GSDME was regulated by the NLRP3 inflammasome-caspase 8-caspase 3 axis and causative for the release of IL-1α. Since cleaved GSDME has previously been reported to form pores in the plasma membrane, it can be assumed that it also enables Th17 cells to release additional, yet to be identified molecules, which are defined by their size or charge (42). This T helper cell associated GSDME expression thus opens up avenues for future research into its regulation and role for human health.

Several of the functions recently assigned to GSDME have been associated with pyroptosis and consecutive enhancement of tumour cell death and of an inflammatory microenvironment (43, 44). Surprisingly, the inventors found that in human Th17 cells, GSDME expression did not translate into pyroptosis. Instead, GSDME expressing Th17 cells displayed preserved viability and continued proliferation upon repetitive TCR stimulation compared to GSDME deficient T cells. The same applied to a comparison of IL-1α-positive and IL-1α-negative T cells. This was unexpected, considering that IL-1 α production has so far been a hallmark of senescent, and thus replication arrested, or of dying cells (45). This evokes the idea that the danger signal IL-1α can be part of a T cell associated cytokine memory that is re-excitable upon cognate antigen recognition (46). The endosomal sorting complexes for transport (ESCRT) mechanisms have recently been proposed as a membrane repair mechanism to preserve cellular integrity upon canonical NLRP3-inflammasome activation and GSDMD mediated pyroptosis (47). Whether an analogous mechanism is operative in human GSDME expressing Th17 cells to preserve viability and a long-term IL-1α cytokine memory, will have to be explored in the future.

The discovery of IL-1α producing human Th17 cells as well as their molecular regulation prompted the question about their pathogenic involvement in autoinflammation. The analysis of three Schnitzler syndrome patients revealed increased IL-1α production by Th17 cell clones from all patients compared to healthy control blood donors. Treatment with the IL-1β blocking monoclonal antibodies, which was accompanied by resolution of clinical symptoms, resulted in normalization of Th17 cell derived IL-1α production. This in vivo effect of IL-1β blockade on Th17 cell specific IL-1α secretion is in line with the observed increase of IL-1α production by IL-1β in T cells in vitro. Although a rigorous causal relationship between IL-1α producing Th17 cells and the pathogenesis of the Schnitzler syndrome and therapy response remains to be established, the data still demonstrate that T cells, which carry an immunological memory and antigen specificity, also contribute to production of the culprit IL-1 cytokine for autoinflammatory syndromes and possibly other chronic inflammatory diseases.

A striking observation was that IL-1α production by T cells is hard-wired through their TCR specificity. While IL-1α production by innate cellular sources is triggered by nonspecific stress stimuli (Gross, O. et al. Inflammasome activators induce interleukin-1 alpha secretion via distinct pathways with differential requirement for the protease function of caspase-1. Immunity 36, 388-400 (2012)), the inventors found IL-1α production by human Th17 cells to be associated with a TCR specificity for C. albicans. Although the TCR repertoire in Th17 cells is also enriched with specificity for S. aureus antigens, S. aureus-specific Th17 cells displayed significantly reduced IL-1α production. These findings are consistent with the differential requirement of IL-1b for the generation of C. albicans- but not S. aureus-specific Th17 cells, as previously reported (4), and, accordingly, the critical role of IL-1b for the induction of IL-1a expression, as reported herein. Additionally, the inventors found IL-1a secretion to be dependent on TCR stimulation and calcium signals, stressing its tight association with specific adaptive immune signaling via the TCR.

Th17 cells are known to be the protagonists for the clearance of C. albicans infections through their secretion of IL-17, which is exemplified by C. albicans dysbiosis in settings of genetic or therapeutic IL-17 deficiencies (48). Strikingly, the inventors found the Th17-cell product IL-1α to be involved in C. albicans clearance because its absence in Th17-cell supernatants significantly reduced C. albicans phagocytosis by monocytes. This suggests that antifungal Th17 cell effector functions are exerted not only through IL-17A/F, as previously suggested, but also through IL-1a in a TCR-specific manner. Whether aberrant regulation of the molecular pathway leading to IL-1a production by Th17 cells could predispose to compromised anti-fungal host defense, will therefore need to be tested in the future.

Cumulatively, the inventor's findings pave the way for a systematic investigation of the contributions of IL-1a-producing Th17 cells in various inflammatory diseases and in antifungal host defence. The TCR-NLRP3 inflammasome-caspase-8 caspase-3-GSDME axis not only represents a previously overlooked mode of immune signalling and fate instruction in Th cells but also provides molecular targets to either disrupt a pathogenic Th17-cell identity or to harness it for host defence.

The present invention relates to the following items.

Item 1. A method for diagnosing an inflammatory disease in a human patient, comprising detecting IL-1α producing Th17 cells in a sample comprising T cells obtained from said patient comprising detecting gasdermin E protein expression, wherein the presence of said IL-1α producing Th17 cells is indicative for an inflammatory disease in the human patient.

Item 2. The method according to Item 1, wherein the IL-1α as produced by the Th17 cells is secreted.

Item 3. The method according to Item 1 or 2, wherein the detection of the gasdermin E protein expression comprises detection of gasdermin E protein pore formation.

Item 4. The method according to any one of Items 1 to 3, further comprising detecting at least one marker selected from NLRP3 inflammasome formation, calpain activity, caspase-3 activity, and caspase-8 activity in said IL-1α producing Th17 cells.

Item 5. The method according to any one of Items 1 to 4, wherein the inflammatory disease is selected from the group of an inflammation that is caused or exacerbated by IL-1α producing Th17 cells, inflammation caused or related to danger signal IL-1α; autoinflammatory Schnitzler syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout.

Item 6. The method according to any one of Items 1 to 5, further comprising the step of detecting the relative amount of the IL-1α producing Th17 cells per volume of the sample and/or per overall Th17 cell population in said sample.

Item 7. The method according to Item 6, further comprising the step of comparing the relative amount of the IL-1α producing Th17 cells as detected to a control sample and/or an earlier sample taken from the same patient.

Item 8. A method for diagnosing the status of an inflammatory disease in a human patient, comprising performing the method according to Item 7, and diagnosing an exacerbated state of the inflammatory disease if an increase of the relative amount of the IL-1α producing Th17 cells is detected or a reduced state of the inflammatory disease if a decrease of the relative amount of the IL-1α producing Th17 cells is detected.

Item 9. A method for identifying an anti-inflammatory compound, comprising the steps of: a) contacting at least one anti-inflammatory candidate compound with the pore forming part of human gasdermin E protein (GSDME-N), and b) detecting the inhibition of assembly/pore formation of GSDME-N in the presence of said candidate compound, when compared to the absence of said candidate compound, wherein the inhibition of assembly/pore formation of GSDME-N identifies an anti-inflammatory compound.

Item 10. The method according to Item 9, wherein said method is performed in vitro or in a recombinant cell, such as, for example, a human Th17 cell, optionally lacking the gasdermin E gene.

Item 11. A method for identifying an anti-inflammatory compound, comprising the steps of: a) contacting at least one anti-inflammatory candidate compound with a cell expressing human gasdermin E protein, b) inducing gasdermin E expression in said cell, and c) detecting the inhibition of assembly/pore formation of GSDME in the presence of said candidate compound, when compared to the absence of said candidate compound, wherein the inhibition of assembly/pore formation of GSDME identifies an anti-inflammatory compound.

Item 12. The method according to Item 11, wherein inducing gasdermin E expression in said cell comprises inducing NLRP3 inflammasome formation, calpain activity, caspase-3 activity, and/or caspase-8 activity.

Item 13. The method according to Item 11 or 12, wherein the inhibition of assembly/pore formation of GSDME in the presence of said candidate compound comprises an inhibition of the expression of gasdermin E and/or caspase-3 in said cell, and/or a reduction of the expression and/or secretion of IL-1α of said cell.

Item 14. The method according to any one of Items 11 to 13, wherein the cell is a human Th17 cell.

Item 15. The method according to any one of Items 11 to 14, wherein the candidate compound is selected from the group consisting of a chemical molecule, a molecule selected from a library of small organic molecules, a molecule selected from a combinatory library, a cell extract, in particular a plant cell extract, a small molecular drug, a protein, a protein fragment, a molecule selected from a peptide library, and an antibody or fragment thereof.

Item 16. The method according to any one of Items 11 to 15, wherein said contacting is in vivo or in vitro, in solution or comprises the candidate compound molecule bound or conjugated to a solid carrier.

Item 17. An anti-inflammatory compound as identified according to a method according to any one of Items 9 to 16, or a pharmaceutical composition comprising said anti-inflammatory compound, together with a pharmaceutically acceptable carrier.

Item 18. A method for preventing or treating inflammation in a subject, wherein the inflammatory disease is selected from the group of an inflammation that is caused or exacerbated by IL-1α producing Th17 cells, inflammation caused or related to danger signal IL-1α; autoinflammatory Schnitzler syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout, comprising administering to said subject an effective amount of an anti-inflammatory compound or a pharmaceutical composition comprising said anti-inflammatory compound according to Item 17.

Item 19. The anti-inflammatory compound or the pharmaceutical composition comprising the anti-inflammatory compound according to Item 17 for use in the prevention or treatment of inflammation in a subject, wherein the inflammatory disease is selected from the group of an inflammation that is caused or exacerbated by IL-1α producing Th17 cells, inflammation caused or related to danger signal IL-1α; autoinflammatory Schnitzler Syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout.

Item 20. A method for monitoring an anti-inflammatory treatment or prophylaxis in a subject in need thereof, comprising a) providing an anti-inflammatory treatment or prophylaxis to said subject, comprising administering to said subject an anti-inflammatory compound or pharmaceutical composition according to Item 17, b) detecting the amount of IL-1α producing Th17 cells in a biological sample obtained from said subject according to a method according to Item 6, and c) comparing the amount(s) as detected in step b) with the amount in an earlier sample taken from said subject, and/or a control sample.

Item 21. A method for predicting or prognosing the success of, progress of and/or sensitivity for an anti-inflammatory treatment or prophylaxis in a subject, comprising providing an anti-inflammatory treatment or prophylaxis to said subject, comprising administering to said subject an anti-inflammatory compound or pharmaceutical composition according to Item 17, performing the method according to Item 10, and detecting the amount of IL-1α producing Th17 cells in a biological sample obtained from said subject according to a method according to Item 6, wherein a decrease of the amount of the IL-1α producing Th17 cells when compared to an earlier sample taken from said subject, and/or a control sample is indicative for the success of, progress of and/or sensitivity for the anti-inflammatory treatment or prophylaxis in the subject.

Item 21. The method according to any one of Items 18 to 21, wherein the subject further receives a second additional anti-inflammatory prophylaxis or therapy.

Item 22. A method for identifying an inflammation modulating compound, comprising the steps of: a) contacting at least one inflammation modulating candidate compound with the pore forming part of human gasdermin E protein (GSDME-N), and b) detecting the inhibition or increase of assembly/pore formation of GSDME-N in the presence of said candidate compound, when compared to the absence of said candidate compound, wherein the inhibition or increase of assembly/pore formation of GSDME-N identifies an inflammation modulating compound, wherein preferably the method is performed in vitro or in a recombinant cell, such as, for example, a human Th17 cell, optionally lacking the gasdermin E gene.

Item 23. A method for identifying an inflammation modulating compound, comprising the steps of: a) contacting at least one inflammation modulating candidate compound with a cell expressing human gasdermin E protein, b) inducing gasdermin E expression in said cell, and c) detecting the inhibition or increase of assembly/pore formation of GSDME in the presence of said candidate compound, when compared to the absence of said candidate compound, wherein the inhibition or increase of assembly/pore formation of GSDME identifies an inflammation modulating compound.

Item 24. The method according to Item 22 or 23, wherein the modulation is inhibition of assembly/pore formation of GSDME and the compound is an anti-inflammatory compound.

Item 25. The method according to Item 23 or 24, wherein inducing gasdermin E expression in said cell comprises inducing NLRP3 inflammasome formation, calpain activity, caspase-3 activity, and/or caspase-8 activity.

Item 26. The method according to any one of Items 23 to 25, wherein the inhibition of assembly/pore formation of GSDME in the presence of said candidate compound comprises an inhibition of the expression of gasdermin E and/or caspase-3 in said cell, and/or a reduction of the expression and/or secretion of IL-1α of said cell.

Item 27. The method according to any one of Items 23 to 26, wherein the cell is a human Th17 cell.

Item 28. The method according to any one of claims 22 to 27, wherein the candidate compound is selected from the group consisting of a chemical molecule, a molecule selected from a library of small organic molecules, a molecule selected from a combinatory library, a cell extract, in particular a plant cell extract, a small molecular drug, a protein, a protein fragment, a molecule selected from a peptide library, and an antibody or fragment thereof.

Item 29. The method according to any one of Items 23 to 28, wherein said contacting is in vivo or in vitro, in solution or comprises the candidate compound molecule bound or conjugated to a solid carrier.

Item 30. An inflammation modulating compound as identified according to a method according to any one of claims 22 to 29, or a pharmaceutical composition comprising said anti-inflammatory compound, together with a pharmaceutically acceptable carrier.

Item 31. A method for preventing or treating inflammation in a subject, wherein the inflammatory disease is selected from the group of an inflammation that is caused or exacerbated by IL-1α producing Th17 cells, inflammation caused or related to danger signal IL-1α; autoinflammatory Schnitzler syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout, comprising administering to said subject an effective amount of an anti-inflammatory compound or a pharmaceutical composition comprising said anti-inflammatory compound according to Item 30.

Item 32. The inflammation modulating compound or the pharmaceutical composition comprising the inflammation modulating compound according to Item 31 for use in the prevention or treatment of inflammation in a subject, wherein the inflammatory disease is selected from the group of an inflammation that is caused or exacerbated by IL-1α producing Th17 cells, inflammation caused or related to danger signal IL-1α; autoinflammatory Schnitzler syndrome, autoinflammatory disorder adult-onset Still's disease (AOSD), systemic-onset juvenile idiopathic arthritis; the syndrome of periodic fever with aphthous stomatitis, pharyngitis, and cervical adenitis (PFAPA); Behçet disease; chronic recurrent multifocal osteomyelitis (CRMO), chronic obstructive pulmonary disorder (COPD); infection, such as fungal infection, inflammation of the lung caused by smoking, and gout.

Item 33. A method for monitoring an inflammation modulating treatment or prophylaxis in a subject in need thereof, comprising a) providing an inflammation modulating treatment or prophylaxis to said subject, comprising administering to said subject an inflammation modulating compound or pharmaceutical composition according to Item 30, b) detecting the amount of IL-1α producing Th17 cells in a biological sample obtained from said subject according to a method according to Item 5, and c) comparing the amount(s) as detected in step b) with the amount in an earlier sample taken from said subject, and/or a control sample.

Item 34. A method for predicting or prognosing the success of, progress of and/or sensitivity for an inflammation modulating treatment or prophylaxis, for example an anti-inflammatory treatment or prophylaxis in a subject, comprising providing an inflammation modulating treatment or prophylaxis to said subject, comprising administering to said subject inflammation modulating compound or pharmaceutical composition according to Item 30, performing the method according to claim 6, and detecting the amount of IL-1α producing Th17 cells in a biological sample obtained from said subject according to a method according to Item 6, wherein a decrease of the amount of the IL-1α producing Th17 cells when compared to an earlier sample taken from said subject, and/or a control sample is indicative for the success of, progress of and/or sensitivity for the inflammation modulating treatment or prophylaxis in the subject.

Item 35. The method according to any one of Items 31 to 34, wherein the subject further receives a second additional inflammation modulating prophylaxis or therapy.

The present invention will now be further described in the following examples and with reference to the accompanying figures and the sequence listing, without being limited thereto. For the purposes of the present invention, all references as cited herein are incorporated by reference in their entireties.

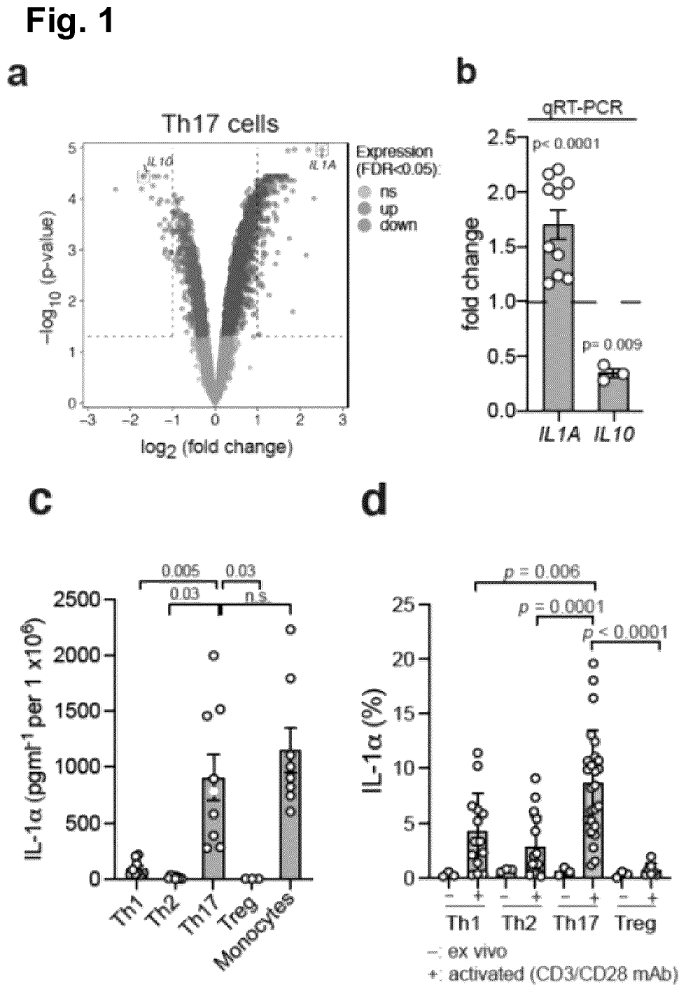

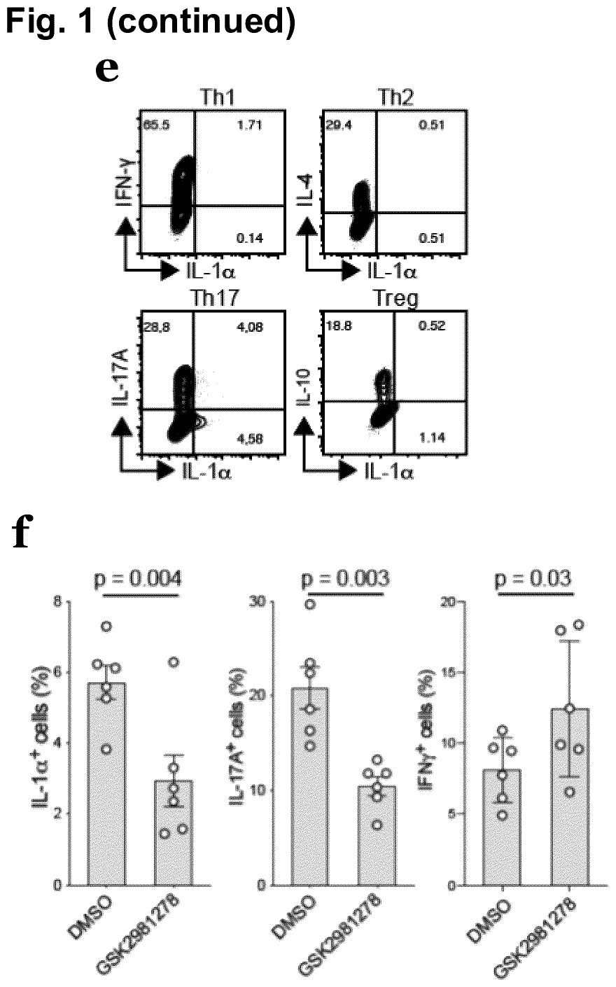

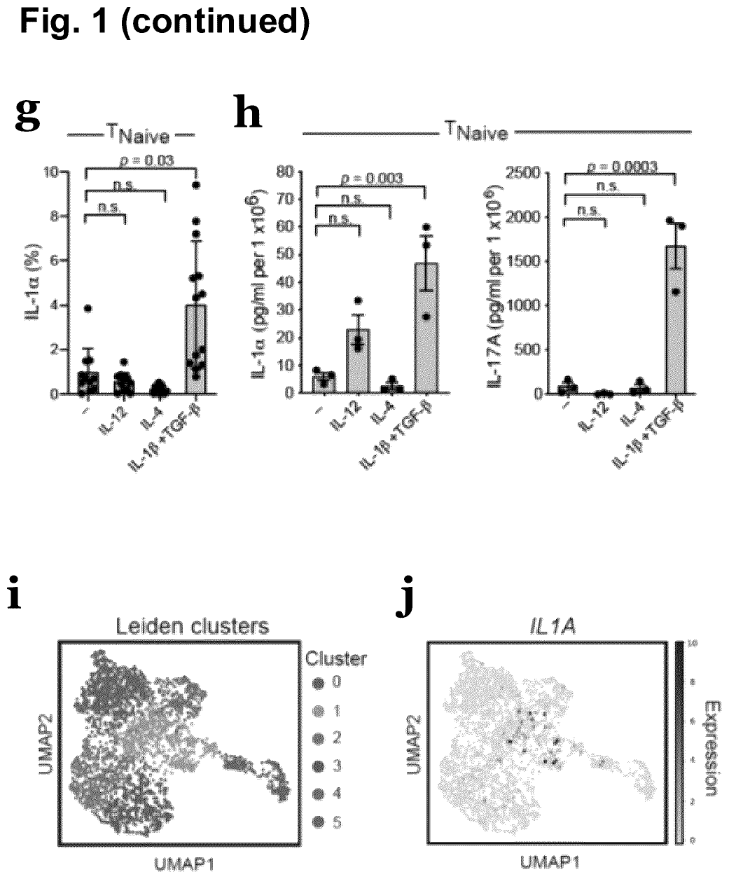

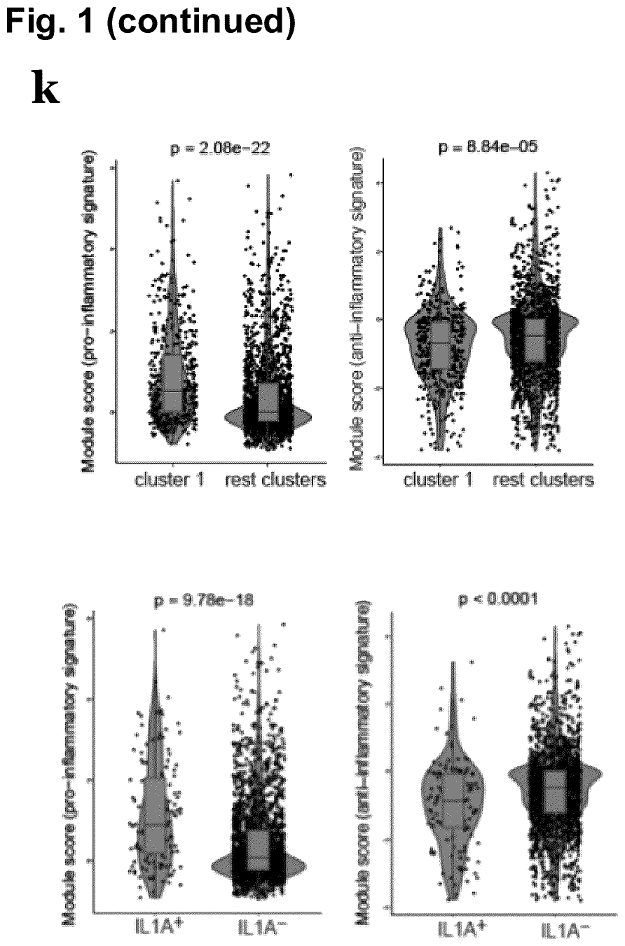

FIG. 1 shows that IL-1α expression is restricted to a proinflammatory subset of human Th17 cells. a) Transcriptome analysis and differentially expressed genes (DEGs) (red, upregulated; blue, downregulated; gray, non-significant genes) of Th17 cells after 5 days of polyclonal stimulation in the presence or absence of IL-1β. b) qRT-PCR analysis of human Th17 cells stimulated as in a). c) ELISA of cell culture supernatants after stimulation of T helper cell subsets as in a). Monocytes were stimulated with LPS for 24 h and nigericin for the last 30 min. One-way ANOVA. (d-g) Intracellular cytokine staining and flow cytometric analysis of T helper cell subsets (d, one-way ANOVA), Th17 cells (c and f, paired student's t test) or naïve T cells (g, one-way ANOVA). h) ELISA with cell culture supernatants from cells stimulated as in (H). i) Leiden clustering in UMAP of Th17 cells. j) single-cell RNA-seq and UMAP of Th17 cells. k) Expression of gene sets in Th17 cells analyzed by singe-cell RNA-seq after either Leiden clustering or grouping into IL1A-positive and IL1A-negative Th17 cells. Wilcoxon rank-sum test with Bonferroni correction.

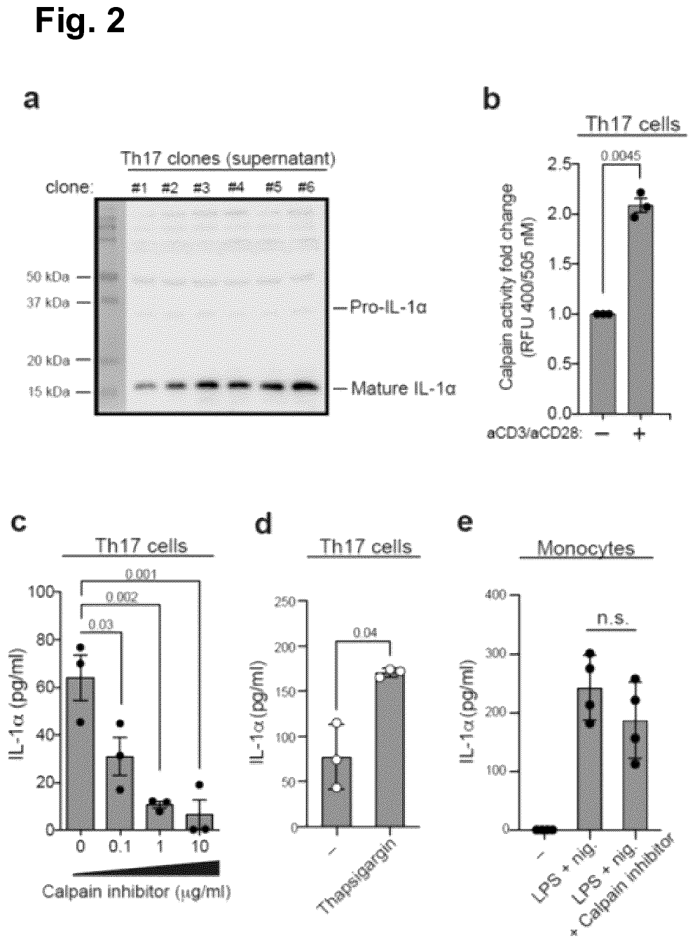

FIG. 2 shows that calpain cleavage is a prerequisite for the release of mature IL-1α by human Th17 cells. (a) Western blot analysis of cell culture supernatants derived from Th17 cell clones that were restimulated with anti-CD3 and anti-CD28 mAb for 5 days. (b) Fold change in relative fluorescence units (RFU) after 1 hour incubation of Th17 cells with the calpain substrate Ac-LLY-AFC. Th17 cells were stimulated for 3 days with anti-CD3 and anti-CD28 mAbs. (c-e) ELISA of cell culture supernatants after stimulation of Th17 cells (C and D) with anti-CD3 and anti-CD28 mAbs (for 5 days) and of monocytes (E) with LPS (24 h) and nigericin (30 min). One-way ANOVA (C and E), paired Student's t test (B and D).

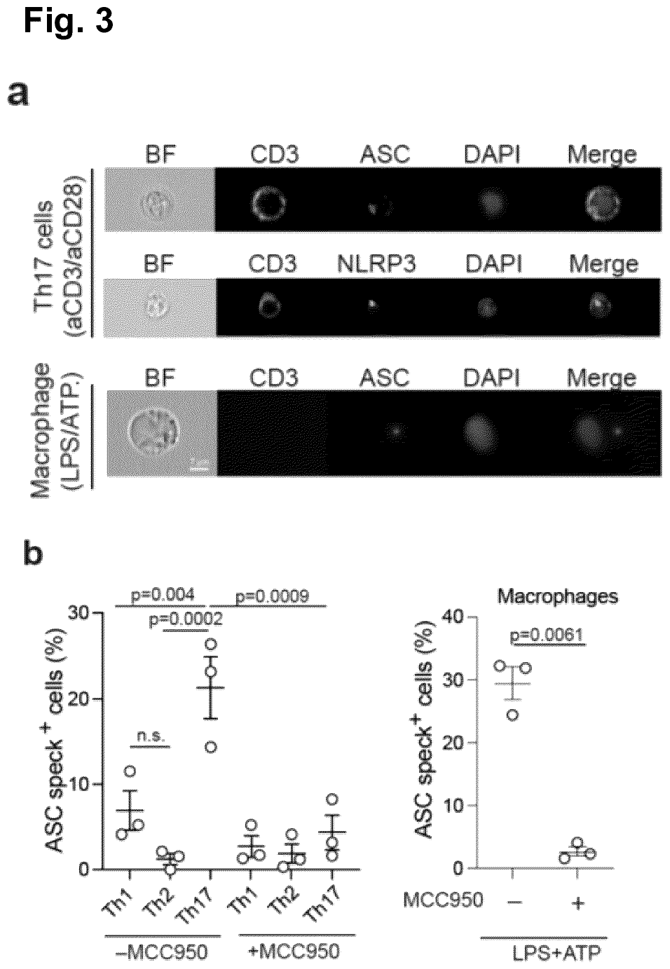

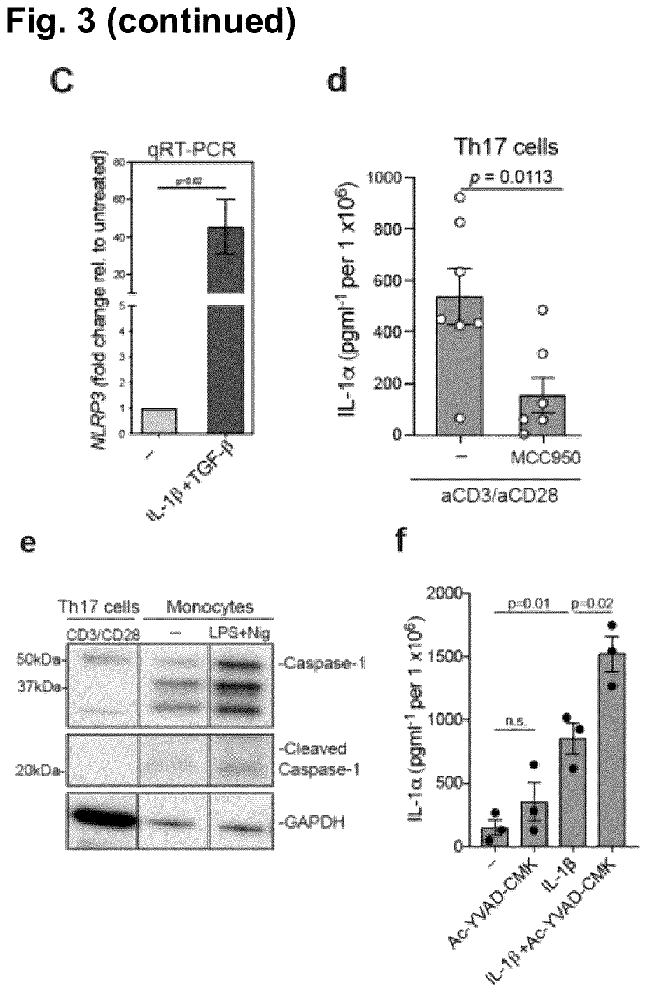

FIG. 3 shows that unconventional NLRP3 inflammasome activation in human Th17 cells regulates IL-1α production. (a, b) Imaging flow cytometry with Th17 cells on day 5 after stimulation with plate-bound anti-CD3 and anti-CD28 mAbs and of macrophages after 24 h of stimulation with LPS and with ATP for the last 30 min. (A) representative experiment. BF, brightfield. (b) cumulative data. Left, One-way ANOVA. Right, paired Student's t test. (c) qRT-PCR analysis of Th17 cells stimulated as in (A) plus restimulation with PMA and ionomycin for 3 hours before RNA extraction. Paired Student's t test. (d) ELISA of cell culture supernatants after stimulation of Th17 cells for 5 days with anti-CD3 and anti-CD28 mAbs. Paired student's t-test. (c) Western blot of cell lysates from Th17 cells after 5 days of stimulation with anti-CD3 and anti-CD28 mAbs and of monocyte lysates after stimulation with LPS for 24 h and with nigericin (Nig.) added for the last 30 min. (f) ELISA as in (E) in the presence or absence of the caspase-1 inhibitor Ac-YVAD-CMK and IL-1β. Paired Student's t test.

FIG. 4 shows that IL-1α exits Th17 cells through gasdermin E pores, which are induced by the NLRP3 inflammasome-casp8-casp3 cleavage cascade. (a) Differential gene expression determined by transcriptome analysis of Th17 cells treated as in FIG. 1A. (b) qRT-PCR analysis of anti-CD3 and anti-CD28 mAb stimulated naïve T cells in polarizing cytokine conditions. (c) Western blot analysis with cell lysates from Th17 cells stimulated with anti-CD3 and anti-CD28 mAb for different durations. The data are representative of 3 experiments. (d) ELISA of cell culture supernatants from Th17 cells with and without deletion of GSDME by Crispr/Cas9 technology. Individual experiments were normalized to the first timepoint of analysis on day 2. n=3, two-way ANOVA. (c) CytoTox 96® Non-Radioactive Cytotoxicity Assay with day 10 supernatants from Th17 cells cultured as in (D). (f) Western blot analysis of cell lysates from Th17 cells stimulated with anti-CD3 and anti-CD28 mAbs for the indicated time points and of CD14⁺ monocytes stimulated for 24 h with LPS and 30 min with nigericin (Nig.). (g) Lane view of electropherograms obtained with a Jess Simple Western System for cell lysates of Th17 cells stimulated for 5 days as in (F) in the presence or absence of the indicated inhibitors. Representative experiment. (h) Cumulative data of (G). one-sample t-test. (i) Luminex assay of the supernatants of Th17 cells stimulated with plate-bound anti-CD3 (1 μg/ml, TR66) and phorbol-12-13-dibutyrate for 8 h on day 4 of culture. (j) ELISA with supernatants of Th17 cells stimulated as in (G). (I, j) paired Student's t test.

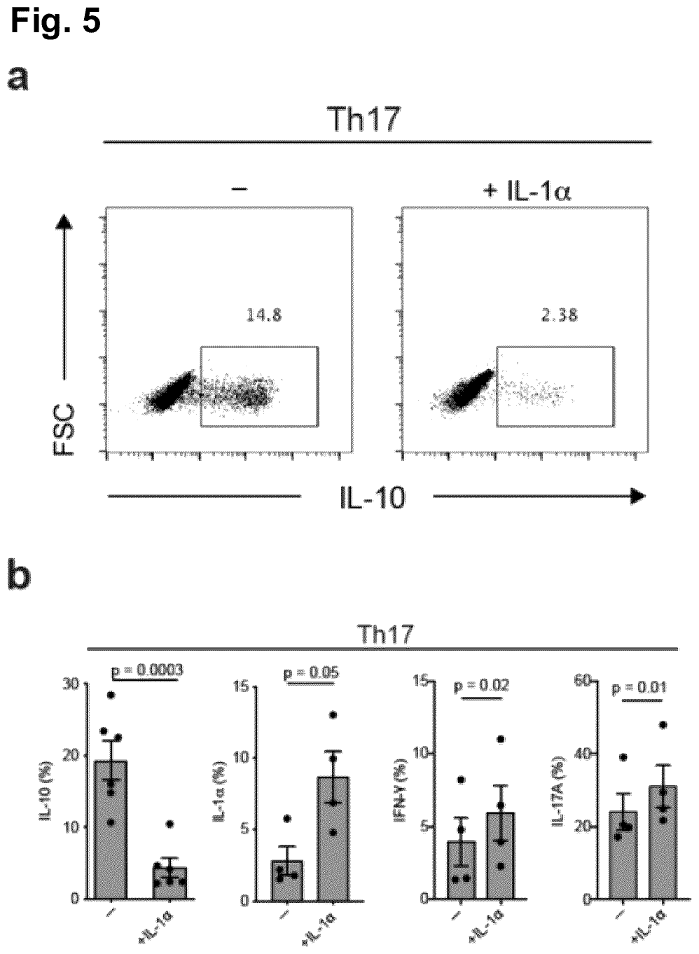

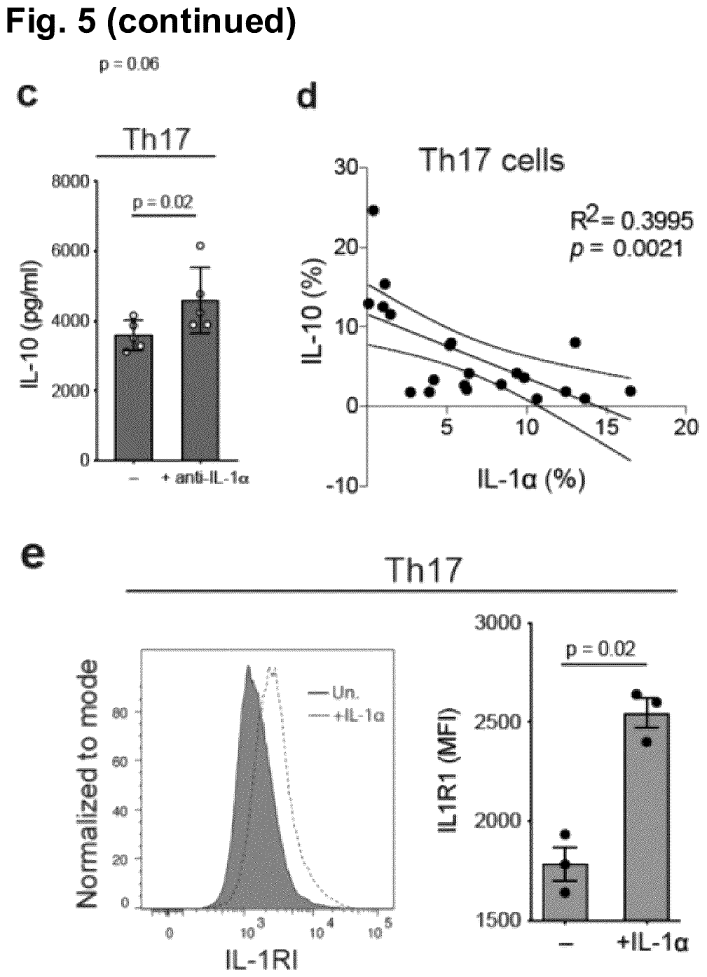

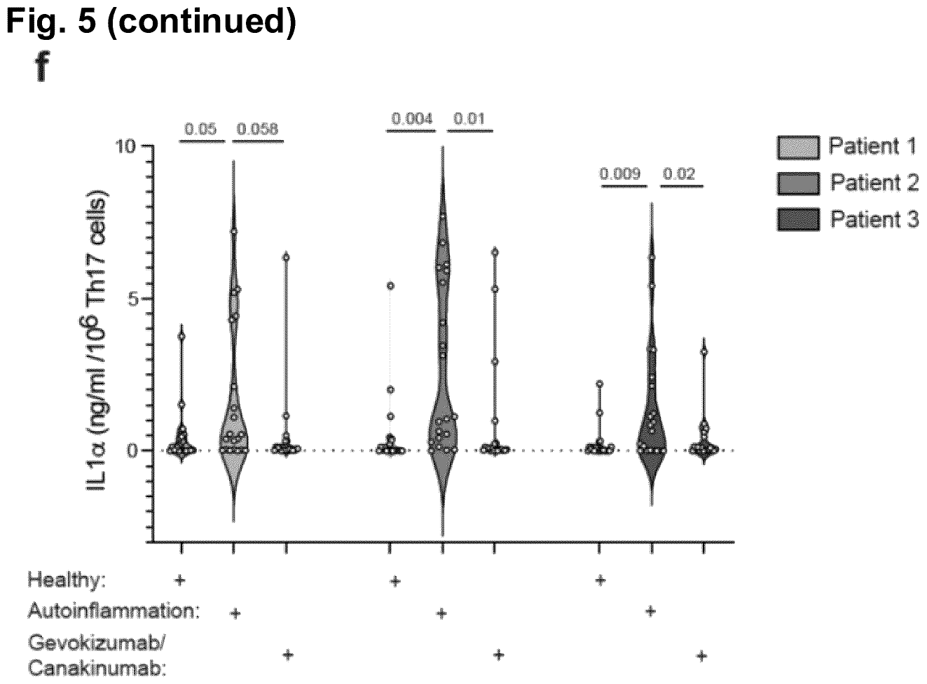

FIG. 5 shows that autocrine IL-1α production by Th17 cells enforces a pathogenic feedback loop and is associated with the autoinflammatory Schnitzler syndrome. (a) Th17 cells were stimulated with anti-CD3 and anti-CD28 mAb for 5 days before intracellular cytokine staining and flow cytometry following PMA and ionomycin restimulation. Representative experiment. (b) Cumulative data for (A). (c) ELISA of supernatants from Th17 cells stimulated for 5 days with anti-CD3 and anti-CD28 mAb. (d) Intracellular cytokine staining and flow cytometric analysis of Th17 cells stimulated for 5 days with anti-CD3 and anti-CD28 mAb. (e) Flow cytometry of Th17 cells stimulated as in (A). (f) Luminex assay of supernatants from Th17 cell clones harvested on day 5 after restimulation with anti-CD3 and anti-CD28mAbs. Th17 cell clones were derived from 3 patients with Schnitzler syndrome before and after therapy (gevokizumab/canakinumab) and from 3 age and sex matched healthy controls. (B, C, E) Paired student's t test. (F) one-way ANOVA.

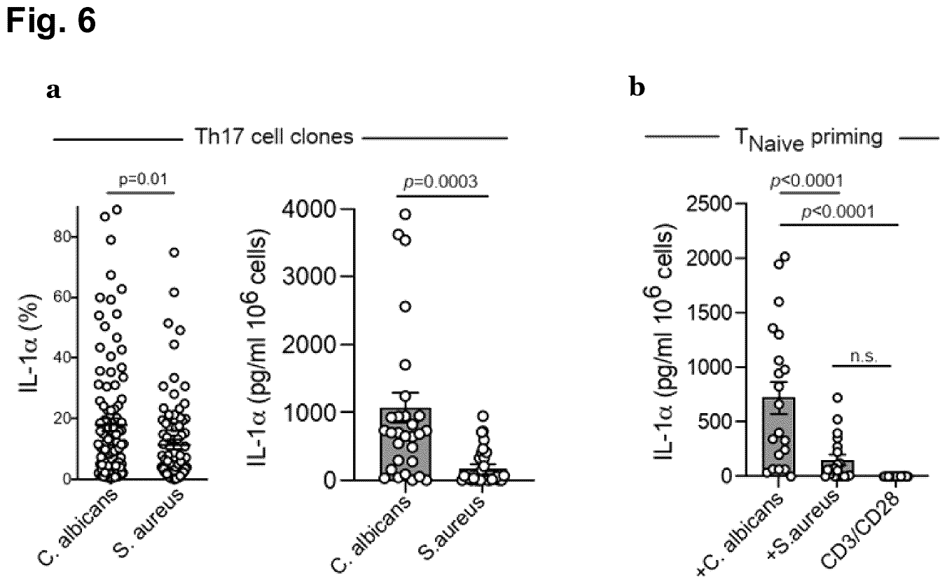

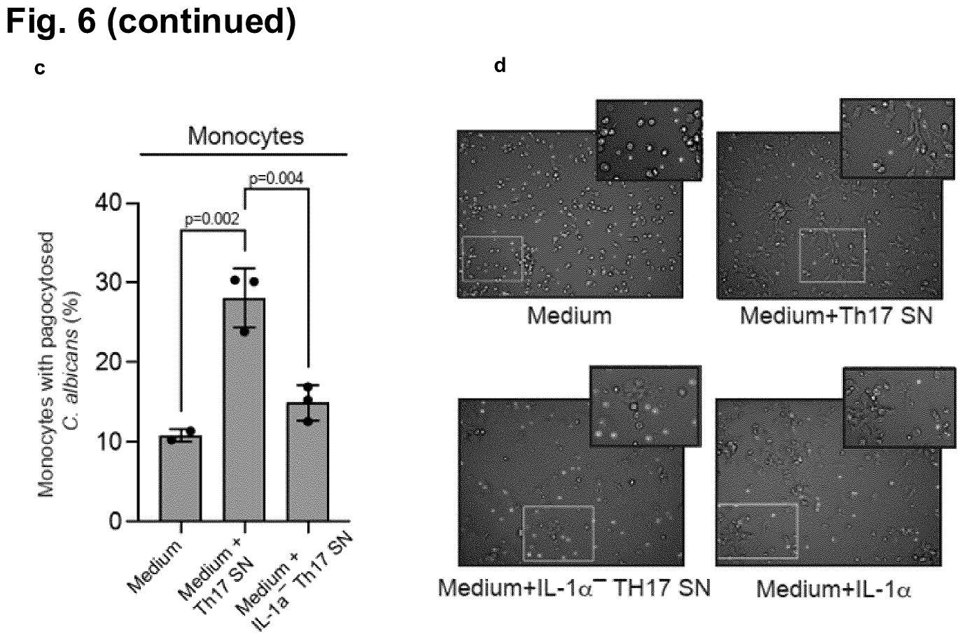

FIG. 6 shows that IL-1a production by human Th17 cells is determined by TCR specificity and contributes to C. albicans clearance. a, Intracellular cytokine staining and flow cytometry (left panel) and ELISA (right panel) of C. albicans versus S. aureus specific Th17 cell clones 14 days after single-cell Th17 cell cloning with irradiated feeder cells and restimulation for 5 days with anti-CD3 and anti-CD28 mAb. The microbial antigen-specific Th17 cells were isolated for subsequent cloning as CFSE-negative population following their restimulation with microbe-pulsed autologous monocytes. Paired Student's t test. b, Naïve Th cells were stimulated with autologous monocytes, which had been pulsed with either C. albicans or S. aureus for 3 h or polyclonally with anti-CD3 and anti-CD28 mAbs. After 5 days of priming, T cells were separated by FACS sorting and restimulated with anti-CD3 and anti-CD28 for 5 days for collection of supernatants for ELISA. One-way ANOVA. c, Flow cytometric analysis of phagocytosis of FITC-labelled heat-inactivated C. albicans yeast by monocytes preincubated for 18 h with IL-1a replete or depleted (immunoabsorption or CRISPR-Cas9 KO) Th17 cell supernatants. One-way ANOVA. d, Real-time live cell in vitro imaging (videos in Extended data Video 1, timepoints) of monocytes in coculture with FITC-labelled C. albicans as in (c). Shown are representative snap shots with magnifications of the videos at timepoint 2 h after addition of C. albicans.

### EXAMPLES

### Cell Purification and Sorting

Peripheral blood mononuclear cells (PBMCs) were isolated by density gradient centrifugation using Ficoll-Paque Plus (GE Healthcare). CD4⁺ T cells were isolated from fresh PBMC by positive selection with CD4-specific MicroBeads (Miltenyi Biotec) using an autoMACS Pro Separator. T helper (Th) cell subsets were sorted to at least 98% purity as follows: Th1 subset, CXCR3⁺CCR4⁻CCR6⁻CD45RA⁻CD25⁻CD14⁻; Th2 subset, CXCR3⁻CCR4⁺CCR6⁻CD45RA⁻CD25⁻CD14⁻; Th17 subset, CXCR3⁻CCR4⁺CCR6⁺CD45RA⁻CD25⁻CD14⁻ as described before (Ghoreschi, K. et al. Generation of pathogenic T(H)17 cells in the absence of TGF-beta signalling. Nature 467, 967-971, doi: 10.1038/nature09447 (2010), Aschenbrenner, D. et al. An immunoregulatory and tissue-residency program modulated by c-MAF in human TH17 cells. Nat Immunol 19, 1126-1136, doi: 10.1038/s41590-018-0200-5 (2018), Noster, R. et al. IL-17 and GM-CSF expression are antagonistically regulated by human T helper cells. Sci Transl Med 6, 241ra280, doi: 10.1126/scitranslmed.3008706 (2014)). Memory Th cells were isolated as CD3⁺CD14⁻CD4⁺CD45RA⁻ lymphocytes, naïve T cells were isolated as CD3⁺ CD14⁻CD4⁺CD45RA⁺CD45RO⁻CCR7⁺ lymphocytes to a purity of over 98%. Cells were sorted with a BD FACSAria™ III (BD Biosciences) or with a BD FACSAria™ Fusion (BD Biosciences). Ethical approval for the use of healthy control and patient PBMCs was obtained from the Institutional Review Board of the Technical University of Munich (195/15s, 491/16 S, 146/17S), the Charité-Universitätsmedizin Berlin (EA1/221/11), the Friedrich Schiller University Jena (2020-1984_1) and the local ethics committee of the Radboud University Medical Center, Nijmegen. The characteristics of patients suffering from Schnitzler syndrome have been described previously (Noster, R. et al. Dysregulation of proinflammatory versus anti-inflammatory human TH17 cell functionalities in the autoinflammatory Schnitzler syndrome. J Allergy Clin Immunol 138, 1161-1169 e1166, doi: 10.1016/j.jaci.2015.12.1338 (2016).). All experiments involving humans were carried out in accordance with the Declaration of Helsinki.

### Cell culture

Human T cells were cultured in RPMI 1640 medium supplemented with 1% (v/v) GlutaMAX™ Supplement, 1% (v/v) MEM nonessential Amino Acids Solution (100×), 1% (v/v) sodium pyruvate (100 mM), 0.1% 2-Mercaptoethanol (50 mM) (all from Gibco™), 1% (v/v) Penicillin-Streptomycin (Sigma-Aldrich), penicillin (500 U/ml), streptomycin (500 μg/ml), and 10% (v/v) fetal calf serum (Sigma-Aldrich). In some experiments, T cell culture was performed in the presence of recombinant cytokines (IL-6, 50 ng/ml; IL-12, 10 ng/ml; IL-4, 10 ng/ml; TGF-b, 10 ng/ml; IL-1b, 20 ng/ml; all from R&D Systems) or neutralizing antibodies (anti-IL-1a, 10 mg/ml, BD Biosciences). Cell cultures were supplemented with the following pharmacological inhibitors where indicated: Z-IETD-FMK (40 μM, R&D Systems), Z-DEVD-FMK (40 μM, R&D Systems), MCC950 (10 μM, R&D Systems), calpain inhibitor II N-Acetyl-L-leucyl-L-leucyl-L-methioninal (0.1-10 μg/ml, R&D Systems), thapsigargin (1 mM, EMD Millipore), Ac-YVAD-CMK (50 μM, R&D Systems), GSK2981278 (10 μM, Cayman Chemical). T cells were stimulated with plate-bound anti-CD3 (2 μg/ml, clone TR66) and anti-CD28 mAbs (2 μg/ml, clone CD28.2; both from BD Biosciences) for 48 h before transfer into uncoated wells for another 3 days for a total culture period of 5 days, unless indicated otherwise in the legends. T cell clones were generated in non-polarizing conditions as described previously following single-cell deposition with fluorescence-activated cell sorting or by limiting dilution cloning (Gross, O. et al. Inflammasome activators induce interleukin-1alpha secretion via distinct pathways with differential requirement for the protease function of caspase-1. Immunity 36, 388-400, doi: 10.1016/j.immuni.2012.01.018 (2012)). Human monocytes were isolated from PBMCs by positive selection with CD14-specific MicroBeads (Miltenyi Biotec). Cells were stimulated with or without 1 μg/mL ultrapure lipopolysaccharide (LPS)-EB (t1r1-3pelps, InvivoGen) for 24 h and nigericin (10 μg/mL, InvivoGen) or ATP (5 mM, Thermo Fisher Scientific) for the last 30 mins. In some experiments, CD14⁺ magnetic activated cell sorting (MACS)-sorted monocytes were differentiated into macrophages for 7 days in the presence of GM-CSF (R&D Systems).

### LDH Assay

Lactate dehydrogenase (LDH) activity was determined with a CytoTox 96® Non-Radioactive Cytotoxicity Assay (G1780, Promega). In short, the supernatants were collected from cells stimulated for 24 hours in RPMI 1640 medium without phenol red (Gibco). Relative LDH release was calculated as follows: LDH release [%]=100×(experimental LDH release (OD₄₉₀)−unstimulated control (OD₄₉₀))/(lysis control (OD₄₉₀) −unstimulated control (OD₄₉₀)).

### CRISPR-Cas9 Knockout Cells

Candidate genes were depleted in sorted cells by using the Alt-R CRISPR-Cas9 system (Integrated DNA Technologies, IDT) in sorted cells after activation with plate-bound anti-CD3 and anti-CD28 for 3 days. In brief, crRNA and tracrRNA (both from IDT) were mixed at a 1:1 ratio and heated at 95° C. for 5 min and cooled to room temperature (RT). Then, 44 mM crRNA:tracrRNA duplex was incubated with at a 1:1 ratio with 36 mM Cas9 protein (IDT) for 20 min at RT to form an RNP complex. A total of 5-10×10⁶ activated T cells were washed with PBS and resuspended in 10 ml of R buffer (Neon transfection kit, Invitrogen). The RNP complex was delivered into cells with a Neon transfection system (10 μl sample, 1600 V, 10 ms pulse width, 3 pulses) (Thermo Fisher Scientific). The electroporated cells were then immediately incubated with RPMI 1640complete medium with IL-2 (500IU). The following crRNAs were used:

### GTCGGACTTTGTGAAATACG (GSDME) (SEQ ID NO: 1),

### ACGCGCACCCACAAGCGGGA (GSDMD) (SEQ ID NO: 2),

### GTCGGAGGAGATCATCACGC (CAPN1) (SEQ ID NO: 3),

### GGCTTCGAAGACTTCACCGG (CAPN2) (SEQ ID NO: 4),

### GGTAGTAGCAACCAACGGGA (IL1A) (SEQ ID NO: 5), and

GTATTACTGATATTGGTGGG (control sequence, NTC) (SEQ ID NO: 6). Knockout efficiency was evaluated on day 7 after electroporation by immunoblotting or enzyme-linked immunosorbent assay (ELISA).

### Cytokine and Transcription Factor Analyses

Intracellular cytokine and transcription factor staining was performed as described before (Noster, R. et al. IL-17 and GM-CSF expression are antagonistically regulated by human T helper cells. Sci Transl Med 6, 241ra280, doi: 10.1126/scitranslmed.3008706 (2014)). Cells were stained with the following antibodies: anti-IL-1a-PE (364-3B3-14), anti-IL-4-FITC (MP4-25D2 5), anti-IL-17A-Pacific Blue (BL168), anti-IFN-g-APC-Cy7 (4S.B3), anti-IL-10-PE-Cy7 (JES3-9D7), (all from Biolegend), anti-RORgt-APC (AFKJS-9, eBioscience), anti-Ki67-BV421 (Biolegend), and anti-IL-1R1-PE (FAB269P, R&D Systems). Then, they were analyzed with a BD LSRFortessa (BD Biosciences), a CytoFLEX Flow Cytometer (Beckman Coulter) or a MACSQuant analyzer (Miltenyi Biotec). Flow cytometry data were analyzed with FlowJo software (Tree Star) or with Cytobank (Cytobank Inc.). The concentrations of cytokines in cell culture supernatants were measured by ELISA (Duoset ELISA kits from R&D Systems, Human Caspase-1SimpleStep ELISA Kit (Abcam) or by Luminex (eBioscience) according to standard protocols as indicated in the corresponding figure legends. Counting beads (CountBright™ Absolute Counting Beads, Thermo Fisher Scientific) were used to normalize for cell numbers if analysis of cumulative supernatants obtained from 5-day cell cultures was performed.

### IL-1a Secretion Assay

The design of the IL-1a secretion assay was adapted based on a previous report (49). Th17 cells (1×10⁶) were stained with 1 mg/ml sulfo-NHS-LC-biotin (ab145611, Abcam), incubated for 30 min at RT, and then washed three times with PBS (pH 8) supplemented with 100 mM glycine. The final washing of cells was performed with PBS supplemented with 0.5% bovine serum albumin (BSA). Cell surface biotinylation was validated with PE-labelled streptavidin (554061, BD Pharmingen™). Purified anti-human IL-1a antibodies (AF-200-NA, R&D) were labeled with streptavidin using a Lightning-Link Streptavidin Conjugation kit (ab102921, Abcam). For cytokine secretion, cells were stimulated with anti-CD3 and anti-CD28 for 72 h. The cells were collected and labeled with streptavidin-IL-1a and incubated for 24 h on the MACSmix™ tube rotator (Miltenyi Biotec). Recombinant IL-1a (Miltenyi Biotec) was added as a positive control. The cells were then stained with a PE-labeled IL-1a antibody (clone: 364-3B3-14, BioLegend).

### Imaging Flow Cytometry

Data acquisition was performed using an ImageStream®X Mk II imaging flow cytometer (AMNIS®; MERCK Millipore) equipped with the INSPIRE software. Briefly, a 60× magnification was used to acquire images with a minimum of 5,000 cells per sample. The following antibodies were used: anti-ASC-PE (HASC-71, Biolegend). anti-CD3-APC or anti-CD3-FITC (UCTH1, Biolegend), and anti-NLRP3-APC (REA668, Miltenyi Biotec). Data analysis was performed using the IDEAS 6.0 software. A compensation matrix was generated using single-stained cells. Cells that were not in the field of focus, clumped cells and debris were excluded. The IDEAS software was used to design masks to define the properties of the spots. For ASC spots, a size of 1-4 μm and a signal to background ratio of 3.0-5.0 were chosen. The mask was trained on at least ten different images with spot-like structures being clearly visible to refine the cutoff for the signal-to-background ratio. From this “spot mask”, the diameter of the mask was measured, and ASC spots in the range of 1-4 μm were considered as true spots.

### Gene Expression Analysis

For analysis of individual gene expression, a high capacity cDNA reverse transcription kit (Applied Biosystems) was used for cDNA synthesis according to the manufacturer's protocol. Transcripts were quantified by real-time PCR (RT-qPCR) with predesigned TaqMan Gene Expression Assays (IL1A, HS00174092-m1; IL1B, Hs01555410_m1; NLRP3, Hs00918082_m1; CASP1, Hs00354836_m1, CAPN2, Hs00965097_m1; GSDMD, Hs00986739_g1; DFNA5, Hs00903185_m1; 18S, Hs03928990_g1) and reagents (Applied Biosystems). mRNA abundance was normalized to the amount of 18S rRNA and is expressed as arbitrary units (A.U.).

For microarray analysis, total RNA was extracted using an RNA MiniPrep kit (Zymo Research) and hybridized to Human Genome U133 Plus 2 Arrays (Affymetrix) according to a whole-transcriptome Pico Kit. Raw signals were processed with the affy R package (Gautier, L., Cope, L., Bolstad, B. M. & Irizarry, R. A. affy—analysis of Affymetrix GeneChip data at the probe level. Bioinformatics 20, 307-315, doi: 10.1093/bioinformatics/btg405 (2004)) and normalized using the robust multiarray average (RMA) expression measure with background correction and cross-chip quantile normalization. The limma R package (Ritchie, M. E. et al. limma powers differential expression analyses for RNA-sequencing and microarray studies. Nucleic Acids Res 43, c47, doi: 10.1093/nar/gkv007 (2015)) was applied to identify differentially expressed genes using linear model fitting and adjusting for differences between biological replicates. Empirical Bayes statistics were used for the moderation of standard errors, and p values were adjusted with the Benjamini & Hochberg method. A false discovery rate (FDR) smaller than 0.05 and a fold change cutoff of 2 were used to define the differentially expressed genes. For gene set enrichment analysis (GSEA) the top 50 up-(pro-inflammatory, 44 significant DEG) and downregulated (anti-inflammatory, 41 significant DEGs) genes from a transcriptomic comparison of IL-10⁺ and IL-10⁻ Th17 cell clones from a public data set (Aschenbrenner, D. et al. An immunoregulatory and tissue-residency program modulated by c-MAF in human TH17 cells. Nat Immunol 19, 1126-1136, doi: 10.1038/s41590-018-0200-5 (2018).) were selected as gene sets and utilized to interrogate the Th17 cell transcriptomes (microarray) following their stimulation in the presence or absence of IL-1b.

For next-generation mRNA sequencing, resting T cell clones categorized as IL-1a⁺ (>30% IL-1a expression) and IL-1a⁻ (0% IL-1a expression) were restimulated with phorbol-12-myristat-13-acetat (PMA) and ionomycin (both from Sigma-Aldrich) for 3 h. A total amount of 1 μg of RNA per sample was used as the input material for the RNA sample preparations. Sequencing libraries were generated using an NEBNext® Ultra™ RNA Library Prep Kit for Illiumina® (NEB, USA) following the manufacturer's recommendations and index codes were added to attribute sequences to each sample. mRNA was purified from total RNA using poly-T oligo-attached magnetic beads. Fragmentation was carried out by using divalent cations under elevated temperature in NEB Next First Strand Synthesis Reaction Buffer (5×) or by using sonication with a Diagenode bioruptor Pico for fragmenting RNA strands. First-strand cDNA was synthesized using random hexamer primers and M-MuL V Reverse Transcriptase (RNase H-). Second-strand cDNA synthesis was subsequently performed using DNA Polymerase I and RNase H. Remaining overhangs were converted into blunt ends via exonuclease/polymerase activities. After adenylation of the 3′ ends of DNA fragments, NEBNext Adaptors with a hairpin loop structure were ligated to prepare for hybridization. To preferentially select cDNA fragments of preferentially 150-200 bp in length, the library fragments were purified with an AMPure XP system (Beckman Coulter, Beverly, USA). Then 3 μI USER Enzyme (NEB, USA) was used with size-selected, adaptor-ligated cDNA at 37° C. for 15 min followed by 5 min at 95° C. before PCR. Then PCR was performed with Phusion High-Fidelity DNA polymerase, Universal PCR primers and Index (X) Primer. At last, PCR products were purified (AMPure XP system) and library quality was assessed on an Agilent Bioanalyzer 2100 system. Clustering of the index-coded samples was performed on a cBot Cluster Generation System using a PE Cluster Kit cBot-HS (Illumina) according to the manufacturer's instructions. After cluster generation, the libraries were sequenced on an Illumina platform, and paired-end reads were generated (Novogene).

For single-cell RNA sequencing, a library of human Th17 cells, which were sorted ex vivo as CCR6⁺CCR4⁺CXCR3⁻ memory Th cells using fluorescence-activated cell sorting (FACS) and then stimulated with anti-CD3 and anti-CD28 mAbs for 4 days (2 days plate-bound), was constructed with Chromium Next GEM Single Cell 5′ Reagents v2 (Dual Index) (10× Genomics, Inc.). The library was sequenced on an Illumina NovaSeq 6000Sequencing System (Illumina, Inc.) according to the manufacturer's instructions, with 150-bp paired-end dual-indexing sequencing (sequencing depth: 20,000 read pairs per cell). Read alignment and gene counting of single-cell data sets was performed with CellRanger v6.1.1 (10× Genomics, Inc.), using the default parameters and the prebuilt human reference 2020-A (10× Genomics, Inc.) based on Ensembl GRCh38 release 98. The output filtered data were first processed with the Python package scanpy v1.7.2 and then analyzed with the R package Seurat v4.0.4. The total count was normalized to 10,000 reads per cell. Each gene was scaled to unit variance, with values exceeding the standard deviation by 10 being clipped. A KNN graph was constructed with a size of 10 local neighboring data points. UMAP with default settings was applied for dimensionality reduction. Clusters were identified by running the Leiden algorithm with a cluster resolution of 0.4. Differential gene expression analysis was performed using the FindMarkers function with the nonparametric Wilcoxon rank-sum test from the R package Seurat v4.0.4.

Gene sets were established from a public data set following transcriptomic comparison of IL-10 versus IL-10⁺ Th17 cell clones¹¹. For both gene sets an average expression score was calculated for each individual cell using the addModuleScore method from the R package Seurat. Differences between scores were tested with the Wilcoxon rank sum test as implemented in the R package stats.

### Immunoblotting

Cells were lysed in RIPA buffer (50 mM Tris, 150 mM NaCl, 1 mM EDTA, 0.1% NP-40, pH 7.5) containing protease inhibitor (Roche) and PhosphoSTOP Easypack (Roche). The protein concentrations of cell lysates were determined with a Pierce™ BCA Protein Assay Kit (Thermo Fisher Scientific™). Total protein (20-40 mg) was boiled with 4× Laemmli sample buffer (Bio-Rad Laboratories) containing 355 mM 2-mercaptoethanol (Thermo Fisher Scientific) at 99° C. for 10 min. The supernatants and lysates were separated by SDS-polyacrylamide gel electrophoresis (PAGE) and transferred to a PVDF membrane (Bio-Rad Laboratories) by using a Mini-Protean system (Bio-Rad Laboratories) according to the manufacturer's protocol. The following primary antibodies were used for immunoblotting: mouse anti-human caspase-8 (Cell Signaling Technology), rabbit anti-human caspase-1 (Cell Signaling Technology), rabbit anti-human IL-1α (Abcam), mouse anti-human GAPDH (Merck millipore), mouse anti-human b-actin (Cell Signaling Technology), and rabbit anti-human gasdermin E (Abcam), rabbit anti-human caspase-3 (Cell Signaling Technology), mouse anti-human caspase-8 (Cell Signaling Technology), rabbit anti-human gasdermin D (Cell Signaling Technology), rabbit anti-human cleaved gasdermin D (Cell Signaling Technology), rabbit anti-NLRP3 (Cell Signaling Technology). HRP-conjugated anti-mouse and anti-rabbit IgG antibodies (Cell Signaling Technology) were used as secondary antibodies. Immunoreactive bands were detected by Pierce™ ECL Western Blotting Substrate or SuperSignal™ West Femto Maximum Sensitivity Substrate (both from Thermo Scientific™). Chemiluminescence signals were recorded with an Odyssey Imaging system (LI-COR Biosciences) and analyzed on Image Studio™ Lite (LI-COR Biosciences). Image contrast was enhanced in a linear fashion when necessary. Protein lysates were also prepared for automated Western blotting using a Jess System (ProteinSimple) according to the manufacturer's instructions. The following primary and secondary antibodies were used: recombinant rabbit anti-gasdermin E-N-terminal (Abcam), rabbit anti-GSDMD (Cell Signaling Technology), rabbit anti-caspase-3 (Cell Signaling Technology), mouse anti-caspase-8 (Cell Signaling Technology), mouse anti-ASC (Santa Cruz Biotechnology, B-3), mouse anti-NLRP3 (Novus Biologicals, 25N10E9), rabbit recombinant anti-Sodium Potassium ATPase antibody (Abcam), mouse anti-GAPDH (Sigma-Aldrich), mouse anti-b-actin (Cell Signaling Technology), anti-mouse HRP-linked secondary antibody (ProteinSimple), and an anti-rabbit HRP-linked secondary antibody (ProteinSimple).

### Extraction of Plasma Membrane Proteins

Plasma membrane proteins were fractionated with a plasma membrane protein kit (Abcam) according to the manufacturer's protocol. In short, 0.5-1×10⁷ cells were collected, homogenized in an ice-cold Dounce homogenizer (Bellco Glass Inc.) and centrifuged at 700× g for 10 mins. The supernatants were collected and centrifuged at 10,000× g for 30 mins. The supernatants were collected as the cytosol fraction. The pellets were used for further extraction of plasma membrane proteins. The purified plasma membrane proteins were enriched in the upper phase solution (Abcam), whereas the lower phase solution contained the cellular organelle membranes. The lysates generated from different fractions were boiled with 4× Laemmli sample buffer (Bio-Rad Laboratories) and subjected to immunoblotting. A rabbit anti-sodium-potassium ATPase antibody (Abcam) was used for a positive control for plasma membrane proteins.

### Calpain Activity Assay

Cells were harvested and washed with cold PBS. Cells were then resuspended in Extraction Buffer (Abcam) and centrifuged at 13,000× g for 5 min. The protein concentration in the supernatants was measured with a Pierce™ BCA Protein Assay Kit (Thermo Scientific™). 40 μg of total lysate protein was used to perform the calpain activity assay (Abcam) following the manufacturer's instructions. A total of 1-2 μl of active calpain (Abcam) was used as a positive control. 1 μL of the calpain inhibitor Z-LLY-FMK (Abcam) was used for a negative control. The lysates and calpain substrate were incubated at 37° C. for 60 min. The fluorometric signal was detected at excitation/emission wavelengths of 400/505 400/505 nm with a CLARIOstar® plate reader (BMG-Labtech).

### Statistical Analysis

The use of the statistical tests is indicated in the respective figure legends, with the error bars indicating the SEM. P values of 0.05 or less were considered to indicate significance. Analyses were performed using GraphPad Prism 9 or R version 4.1.

### The Production of the Innate Danger Signal IL-1α is a Property of a Human Th17 Cell Subset

Pro- and anti-inflammatory human Th17 cell post-activation fates have previously been identified based on their differential coexpression of IL-10 (4, 7). To reveal the culprits of pathogenicity in the Th17 cell subset, the inventors performed a transcriptomic comparison of Th17 cells, which were activated in the presence or absence of IL-1β, a cytokine, which has previously been demonstrated to confer pathogenicity to Th17 cells by IL-10 suppression (4, 6, 7). IL1A was among the top IL-1 β-upregulated genes (FIG. 1a). This was surprising considering that IL-1 α does not belong to the effector cytokine repertoire of T cells, but instead represents an innate danger signal. As expected, IL10 was among the top downregulated genes in the IL-1β stimulated Th17 cells (FIG. 1a). The reciprocal expression pattern of IL-1α and IL-10 upon IL-1β stimulation was further validated by qRT-PCR in Th17 cells from several independent healthy blood donors (FIG. 1b). Gene set enrichment analysis (GSEA) with the top 50 up- and downregulated genes in IL-10⁺ and IL-10⁻Th17 clones using a public data set (7), underlined the pro-inflammatory transcriptomic identity of the inventors' IL-1β-treated Th17 cells. Together, these data identified IL-1α to be a property of human Th17 cells and to correlate with a pro-inflammatory T cell identity.

To investigate, whether IL-1α expression was a general property of T cells, the inventors enriched individual Th cell subsets from peripheral blood mononuclear cells (PBMCs) by their differential expression of chemokine receptors and compared their IL-1α secretion after 5 days of polyclonal T cell receptor stimulation. IL-1α was specifically produced by the Th17, but not Th1, Th2 and Treg subset (FIG. 1c-e). Strikingly, its secretion level upon stimulation with anti-CD3 and anti-CD28 mAbs matched that of human monocytes stimulated with lipopolysaccharide (LPS) and nigericin, indicating that human Th17 cells, notwithstanding their adaptive immune identity, serve as a major source of the danger signal IL-1α (FIG. 1c). Intracellular IL-1α protein expression was absent in freshly isolated resting Th cells but inducible upon T cell receptor (TCR) activation with significant enrichment in Th17 cells compared to Th1, Th2 and Treg enriched cells (FIG. 1d). Mouse T cells did not produce any IL-1α (data not shown) consistent with previous reports.

The unique association of IL-1α with the Th17 cell subset prompted us to mechanistically dissect its regulation. The Th17 cell identity is regulated by the master transcription factor ROR-γt (15). Interestingly, IL-1α expression was reduced upon specific inhibition of RORγt (FIG. 1e, f). These data are in line with putative binding sites of RORγt and also RORα in the IL1A promotor and enhancer region.

The fate of a particular T helper cell subset is determined by a distinct polarizing cytokine microenvironment upon naïve T cell stimulation. The inventors therefore tested whether the Th17 cell polarizing cytokine combination of IL-1β and TGF-β as compared to the Th1 and Th2 polarizing cytokines IL-12 and IL-4, respectively, would bias the naïve T cell fate towards IL-1α production. Indeed, the inventors observed the highest intracellular expression and secretion of IL-1α upon naïve T cell priming in Th17 polarizing conditions (FIG. 1g, h). Th1 and Th2 priming conditions did, instead, not significantly alter IL-1α secretion compared to naïve T cells that were stimulated in the absence of polarizing cytokines. Together, this demonstrates that naïve T cells acquire the property to produce IL-1α through Th17 polarizing cytokines.

To investigate whether these IL-1a producing Th17 cells constitute a distinct subpopulation within Th17 cells, the inventors performed single-cell RNA-sequencing (scRNAseq) of human Th17 cells following 4 days of polyclonal activation. High-dimensional space by uniform manifold approximation and projection analysis (UMAP) and Leiden clustering of all Th17 cells identified 6 individual clusters (FIG. 1i). A distinct and rare (6.06%) population of IL1A expressing Th17 cells was selectively enriched in cluster 1 (FIG. 1,i, j). This cluster 1 was significantly more pro- and less anti-inflammatory than all other clusters as indicated by GSEA using the pro- and anti-inflammatory gene sets generated from the public transcriptome data for IL-10⁺ and IL-10⁻Th17 cell clones (FIG. 1k). Direct comparison of IL1A positive versus IL1A negative Th17 cells across all clusters confirmed the conclusion that IL1A positive Th17 cells displayed a significantly enhanced proinflammatory and reduced anti-inflammatory signature (FIG. 1k). On the protein level, Th17 cell clones also segregated into IL-1a positive and negative T cell clones, thus supporting heterogeneity of IL-1a protein expression at the single-cell level within the Th17 cell population. Together, this revealed the existence of a distinct subpopulation of IL-1a expressing cells within the Th17 cell subset, which distinguished itself from other Th17 cells by a significantly enhanced proinflammatory transcriptomic signature.

### Calpain Cleavage of Pro-IL-1α is a Prerequisite for IL-1α Release by Th17 Cells Via an Unconventional Secretion Pathway

The mechanism of IL-1α secretion in T cells remains completely unexplored. To test whether the unconventional ER/Golgi-independent secretion pathway (19) was operative for the release of IL-1α in human T cells, as has previously been reported for antigen presenting cells (20), the inventors stimulated Th17 cells for 5 days with CD3 and CD28 mAbs and tested for intracellular IL-1α and IL-17 expression after restimulation with PMA and ionomycin in the presence or absence of the protein transport inhibitor brefeldin A (BFA). In contrast to IL17A expression, intracellular IL-1α expression was not influenced by BFA. Accordingly, the secretion of IL17A, but not IL-1α into the extracellular space was reduced by BFA. Together, these data confirm an unconventional ER/Golgi-independent pathway for IL-1α secretion in human T cells.

While cleavage of pro-IL-1β is required to generate bioactive extracellular IL-1β, IL-1α is known to be passively released upon cell death as an alarmin and to exert its bioactive potential after binding to IL-1RI in its uncleaved or cleaved form (21). To find out whether pro-IL-1α undergoes intracellular processing for a controlled release by human T cells, the inventors determined the full length and cleaved forms of IL-1α in the supernatant of activated Th17 cells by western blotting. To exclude any contaminating monocytes as a potential source of uncleaved IL-1α the inventors generated Th17 cell clones over a period of 2 weeks with CD3 and CD28 mAb and subjected them, after a washing step, to TCR restimulation for another 5 days before western blotting, thus excluding any persistence of the short-lived monocytes. In all six tested Th17 cell clones, the inventors found a preferential enrichment of the cleaved form of IL-1α in the supernatant (FIG. 2a). This excludes passive release of pro-IL-1α during cell necrosis as default IL-1α exit modality in T cells and, instead, highlights that human Th17 cells must possess a molecular machinery for pro-IL-1α cleavage and thus for the controlled release of this potent bioactive molecule by viable T cells in contrast to innate cells.

Several proteolytically active enzymes, including thrombin, granzyme B and calpains, have previously been reported to process pro-IL-1α at distinct cleavage sites (22-24). Calpain is a calcium dependent cysteine protease giving rise to the bioactive p17 fragment that the inventors identified herein (24). The inventors detected calpain activity in Th17 cells, which strongly increased upon their activation with CD3 and CD28 mAbs (FIG. 2b). To test the dependence of IL-1α secretion on T cell intrinsic calpain activity, the inventors applied increasing concentrations of the calpain inhibitor to Th17 cells that were activated with CD3 and CD28 mAb for 5 days. The inventors observed a dose-dependent reduction in IL-1α secretion by Th17 cells into the extracellular space as measured by ELISA (FIG. 2c). IL-1α secretion increased upon intracellular accumulation of calcium upon inhibition of the sarco/endoplasmic reticulum Ca2⁺ ATPase with thapsigargin (FIG. 2d). Instead, LPS/nigericin-induced IL-1a secretion by monocytes was not dependent on calpain (FIG. 2e). To corroborate the dependence of IL-1α secretion on calpain, the inventors also genetically knocked out calpains in human Th17 cells using CRISPR-Cas9. This revealed a role for CAPN2, but not CAPN1, for IL-1a secretion by human Th17 cells. These data are consistent with the preferential expression of CAPN2 but not CAPN1 by human T cell subsets in contrast to dendritic cells, which display a reversed calpain gene expression pattern. Together, these data demonstrate that the proteolytic activity of the calcium dependent protease calpain is a prerequisite for the unconventional IL-1α secretion by TCR-activated human Th17 cells.

### IL-1α Production by Human Th17 Cells is Regulated by the NLRP3 Inflammasome

Despite the essential role of calpain for pro-IL-1α maturation, the mechanism leading to its extracellular release still remains elusive. IL-1β cleavage and release, instead, are known to be regulated by the NLRP3-inflammasome, a multi-molecular platform for caspase-1 activation, which also enables the formation of IL-1β permissive gasdermin D (GSDMD) membrane pores and pyroptosis (26). Even though IL-1α does not possess any cleavage sites for caspase-1, its secretion in myeloid cells has previously been associated with NLRP3-inflammasome activation, non-enzymatic activity of caspase-1 and IL-1β release (14, 27). To assess whether human Th17 cells possess the molecular scaffold of the NLRP3-inflammasome, the inventors tested the expression of NLRP3 and the adaptor molecule apoptosis-associated speck-like protein containing a CARD (ASC) in human Th17 cells. Western Blot analysis confirmed the presence of these inflammasome components. A hallmark of NLRP3-inflammasome activation is the formation of an ASC-speck, a micrometer-sized structure that is formed in the cytoplasm upon assembly of the inflammasome components ASC and NLRP3 for the dynamic recruitment and activation of pro-caspase-1 (28, 29). The inventors found ongoing inflammasome activation in human Th17 cells upon polyclonal stimulation by identification of ASC-specks using the ImageStream technology (FIG. 3a, b). Strikingly, increased frequencies of ASC specks were uniquely confined to the Th17 cell subset (FIG. 3b, left). Th17 cells even approximated the ASC speck formation of LPS and ATP stimulated macrophages (FIG. 3b, right). Th1 and Th2 cell subsets, in contrast, displayed background ASC speck levels (FIG. 3b, left). Moreover, ASC-speck formation was completely abrogated in the presence of the specific NLRP3 inflammasome inhibitor MCC950, substantiating ongoing NLRP3 inflammasome activation in Th17 cells (FIG. 3b, left). The selective engagement of the NLRP3 inflammasome by Th17 cells was further supported by the strong induction of NLRP3 transcripts upon T cell stimulation in the presence of the Th17 cell-polarizing cytokines IL-1β and TGF-β (FIG. 3c). Importantly, IL-1a secretion by Th17 cells was significantly reduced by NLRP3 inflammasome inhibition with MCC950 (FIG. 3d), thus demonstrating the critical role of the NLRP3 inflammasome for the secretion of IL-1α by human Th17 cells.

Caspase-1 is the canonical effector protein in the NLRP3 inflammasome complex. Interestingly, the inventors observed pro-caspase-1 expression in activated human Th17 cells (FIG. 3e). However, in contrast to LPS and nigericin stimulated monocytes, no cleaved caspase-1 was detected in human Th17 cell lysates (FIG. 3e). The inventors further excluded extracellular release of caspase-1 by ELISA. Importantly, pharmacological inhibition of caspase-1 with Ac-YVAD-CMK did not inhibit IL-1a secretion in the presence or absence of the IL-1a stimulus IL-1b. Rather, the inventors even observed a significantly elevated IL-1a secretion upon caspase-1 inhibition (FIG. 3f). In line with the absence of bioactive caspase-1, the inventors did not observe any secretion of IL-1b by human Th17 cells, in contrast to that for LPS and ATP-stimulated monocytes. Furthermore, no intracellular IL-1b was detectable or inducible by IL-1α polarizing cytokines in Th17 cells or Th17 cell clones. This was in contrast to the coexpression of IL-1β and IL-1α at the single-cell level observed in monocytes, consistent with their previously suggested cosecretion and putative coregulation pattern. Cumulatively, these data demonstrate that human Th17 cells produce IL-1α independently of caspase-1 and IL-1β, unlike monocytes and presumably other innate immune cells, despite clear involvement of the NLRP3inflammasome. This finding points to a distinct IL-1α secretion mechanism for T cells.

### Gasdermin E Pores Serve as Conduits for IL-1a Secretion by Human Th17 Cells

Gasdermins belong to a family of recently identified pore-forming effector molecules that enable the release of inflammatory mediators (31). Gasdermin D (GSDMD) is a direct target of caspase-1 and thus regulated by NLRP3-inflammasome activation. However, GSDMD was not upregulated in the proinflammatory Th17 cell subset upon IL-1β treatment as assessed by differential gene expression following transcriptomic analysis (FIG. 4a). Interestingly, the transcriptome analysis revealed instead a selective upregulation of GSDME expression in the pro-inflammatory IL-1β stimulated Th17 cell subset, but no regulation of any other member of the gasdermin family (FIG. 4a). This was surprising considering that gasdermin E (GSDME) has never been reported in T cells before. GSDME has previously been shown to form membrane pores in innate immune cells that may serve as conduits for the extracellular release of alarmins and that may initiate pyroptotic cell death similar to GSDMD. To test the association of GSDME with the Th17 cell subset, the inventors assessed whether Th17 cell polarizing cytokines would coregulate GSDME. Interestingly, only the combination of TGF-β and IL-1β (Th17) but not IL-12 (Th1) or IL-4 (Th2), upregulated GSDME transcripts as assessed by RT-qPCR (FIG. 4b). GSDMD, instead, was not regulated by T cell polarizing cytokines.

This prompted the inventors to test GSDME expression also on the protein level in human Th17 cells. Interestingly, the GSDME pro-form was inducible upon T cell receptor activation. It was expressed as early as 24 h after polyclonal stimulation as assessed by western blotting. The cleaved N-terminal pore forming GSDME was detectable at late time points, 3-4 days after TCR stimulation of Th17 cells (FIG. 4c). Full-length GSDMD was concomitantly induced upon T cell activation. Yet in stark contrast to GSDME, no cleavage of GSDMD was observed as predicted from absence of caspase-1 and IL-1β secretion, leaving its role in Th17 cells open for further analysis (FIG. 4c).

The inventors next aimed to explore whether GSDME pores served as conduits for the extracellular release of IL-1α in Th17 cells. To this end, the inventors first ascertained expression of the cleaved pore-forming N-GSDME unit in the plasma membrane. The inventors then knocked out GSDME by CRISPR-Cas9 and monitored IL-1α release into the supernatant over time by ELISA. Interestingly, absence of GSDME but not of GSDMD significantly inhibited the release of IL-1α by Th17 cells (FIG. 4d).

Surprisingly, GSDME expression by Th17 cells was not associated with pyroptotic cell death as no difference in extracellular LDH concentrations was detected between GSDME deficient or intact Th17 cells (FIG. 4c) or between IL-1α-positive and IL-1α-negatively sorted Th17 cells. IL-1α-positive Th17 cells displayed even higher Ki67expression compared to their IL-1α negative counterparts after 5 days of polyclonal stimulation. This was consistent with an enrichment of a proliferation gene signature when transcriptomes of IL-1α positive Th17 clones were compared to those of IL-1α-negative Th17 cell clones. Finally, IL-1α positive Th17 clones even continued to reexpress IL-1α upon repetitive TCR restimulation cycles demonstrating that the tunnelled release of IL-1α by Th17 cells is maintained upon repetitive restimulations and indicative of a reinducible T cell cytokine memory.

### The Casp8-Casp3-GasderminE Proteolytic Cleavage Cascade Enables IL-1α Secretion Upon NLRP3 Inflammasome Activation in Human Viable Th17 Cells

The inventors next explored the possibility of a mechanistic crosstalk of NLRP3inflammasome activation and GSDME cleavage in human Th17 cells. Different enzymes have recently been attributed roles in the cleavage of GSDME, including caspase-3. Caspase-3 is a target of caspase-8, which, in turn has previously been shown to be recruited by the NLRP3 inflammasome, in particular in settings of caspase-1 deficiency (32, 33). The inventors therefore hypothesized the NLRP3 inflammasome-caspase 8-caspase 3-GSDME axis to be operative for the production of IL-1α by human Th17 cells. Indeed, both pro-caspase-8 and pro-caspase 3 were detectable in Th17 cells. The inventors found that cleavage of both caspases occurred upon TCR stimulation and preceded the cleavage of GSDME (FIG. 4f). No cleaved products of caspase-8 and caspase-3 were detectable in nigericin and LPS stimulated monocytes (FIG. 4f). To establish a causative role of these caspases for the cleavage of GSDME and for IL-1α secretion in T cells, the inventors pharmacologically blocked the caspase-3 or caspase 8 activity with the inhibitors Z-DEVD-FMK or Z-IETD-FMK, respectively. Inhibition of caspase-8 activity with Z-IETD-FMK translated into abrogation of its downstream target caspase-3 in line with early reports (34). Both treatments reduced GSDME cleavage, while also abrogating IL1α secretion (FIG. 4g to j).

These data clearly demonstrated that the caspase 8-caspase 3-GSDME axis was operative in human Th17 cells upon TCR activation. To finally establish the link to the NLRP3 inflammasome, the inventors applied MCC950 to stimulated Th17 cells, which, indeed, revealed a significant reduction in caspase-3 and GSDME cleavage on day 5 (FIG. 4g and h). A reduction of caspase-8 cleavage was less pronounced at the timepoint of analysis, which is, however, in line with its earlier activation time window (FIG. 4f). In sum, the targeted inhibition of each individual molecular player established the NLRP3 inflammasome-caspase 8-caspase 3-GSDME cascade as the proteolytic pathway for the extracellular release of bioactive IL-1α by human Th17 cells.

### Autocrine IL-1a Represses IL-10 Production in Th17 Cells and Increased IL-1a Expression is Associated with the Autoinflammatory Schnitzler Syndrome

After having identified IL-1α as a new effector cytokine of Th17 cells as well as its molecular regulation, the inventors next explored the physiological and clinical relevance of the IL-1α producing Th17 cell subset. Exogenous application of recombinant IL-1α reduced IL-10 expression by Th17 cells (FIG. 5a), while simultaneously promoting the pro-inflammatory Th17 cell phenotype by upregulation of IFN-γ and IL-17A (FIG. 5b). Inversely, abrogation of autocrine IL-1α signalling with IL-1α neutralizing antibodies promoted an anti-inflammatory Th17 cell phenotype through increased IL-10 production upon polyclonal stimulation (FIG. 5c). This was in line with the reciprocal expression pattern of IL-10 and IL-1α within activated Th17 cells (FIG. 5d). Autocrine IL-1α production also induced the upregulation of its own receptor IL-1R1 (FIG. 5e). Together, this suggests that the IL-1α producing Th17 cell subset maintains its pathogenic identity via an autocrine IL-1αIL-10^{down}-IL-1R^{UP} feedback loop independent of an innate exogenous source of Il-1β.

Autoinflammatory syndromes are very rare clinical disorders characterized by recurrent febrile episodes and inflammatory cutaneous, mucosal, serosal and osteoarticular manifestations that have been mechanistically linked to IL-1 overproduction by the innate immune system (35). Given its regulation by the NLRP3 inflammasome, this prompted the question whether the IL-1α-producing Th17 cell subset was also involved in the pathogenesis of this disease entity. The inventors isolated Th17 cells ex vivo from the blood of three independent patients suffering from the rare autoinflammatory Schnitzler syndrome and generated T cell clones, which were restimulated with CD3 and CD28 mAbs for 5 days to assess their IL-1α secretion levels (6). This revealed significantly increased IL-1α production by Th17 cell clones in all patients compared to healthy controls (FIG. 5f). Treatment with the IL-1β-neutralizing antibody gevokizumab or canakinumab for 6 months resulted in a significant reduction of IL-1α production by Th17 cells in all patients to levels found in healthy control blood donors and led to complete resolution of all clinical symptoms in the inventors' patients (6). This demonstrates that T cells, beyond innate immune cells, can also contribute to the production of the culprit disease defining cytokine IL-1α in the autoinflammatory Schnitzler Syndrome and that they are responsive to IL-1β neutralizing therapies in vivo. The identification of GSDME pores in T cells as an exit strategy for proinflammatory IL-1α and their regulation by the NLRP3-inflammasome-caspase 8-caspase 3-GSDME axis could provide a new therapeutic rationale for a range of inflammatory diseases.

### IL-1a Production by Human Th17 Cells is Determined by TCR Specificity and Contributes to Antifungal Host Defence

The inventor's finding that human Th17 cells produce the innate danger signal IL-1a and repurpose an innate signalling machinery for its extracellular release blurs the distinction of adaptive versus innate immune responses and thus extends the overall functional repertoire of T cells. A critical feature, that remains characteristic for adaptive memory responses is TCR-endowed antigen specificity. The inventors therefore investigated whether the ability of human Th17 cells to produce IL-1a is restricted to specific antigen specificities. Th17 cells have previously been shown to be highly enriched with cells specific for C. albicans and S. aureus antigens (16). The inventors therefore tested whether C. albicans-versus S. aureus-specific Th17 cells differed in their ability to produce IL-1a. CFSE-labelled Th17 cells were co-cultured with autologous monocytes, which were pulsed with either heat-killed C. albicans yeast cells or S. aureus lysates as described previously (4, 16). In accordance with previous reports (4, 16), the inventors observed robust proliferation of a significant proportion of Th17 cells but not of other Th cell subsets in response to each of these microbial antigens, respectively. The CFSE-negative single Th17 cells were then cloned on day 7 and tested after 14 days for their ability to express IL-1a on the single-cell level by flow cytometry or to secrete IL-1a into the supernatant. Interestingly, the inventors observed significantly greater IL-1a expression and secretion by C. albicans-specific than by S. aureus-specific Th17 cell clones (FIG. 6a). The inventors next investigated whether naïve T cells recognizing C. albicans rather than S. aureus, would be primed by their cognate antigen to selectively produce IL-1a. Naïve (CD45RA⁺CCR7⁺) Th cells were cocultured with autologous monocytes pulsed with heat-inactivated C. albicans or S. aureus antigens or stimulated polyclonally with anti-CD3 and anti-CD28 mAbs, and then cloned on day 7 with allogeneic feeder cells. All clones were restimulated on day 14 with anti-CD3 and anti-CD28 mAbs for 5 days for ELISA of their supernatants. Strikingly, the inventors found that C. albicans, but not S. aureus or polyclonal TCR activation, induced de novo IL-1a production in human differentiating Th17 cells (FIG. 6b). The inventors' combined ex vivo recall and in vitro priming approach therefore establishes that the ability to produce IL-1a is confined to T cells with TCR specificity for C. albicans.

The inventors finally tested whether the distinctive ability of C. albicans-specific Th17 cells to produce IL-1a is associated with a physiological role in anti-fungal host defence. For this, the inventors cocultured human monocytes with supernatants from human Th17 cells following their polyclonal restimulation with anti-CD3 and anti-CD28 mAbs for 5 days. The inventors observed significantly increased phagocytosis of FITC-labelled C. albicans by monocytes using flow cytometry. Importantly, the increased C. albicans phagocytosis by Th17 cell supernatants was IL-1a dependent as shown by significant abrogation of C. albicans phagocytosis if Th17 cell supernatants were devoid of IL-1a following immunoabsorption or CRISPR-Cas9 targeted IL-1a depletion in Th17 cells (FIG. 6c). Live cell in vitro imaging further corroborated the increased uptake and elimination of C. albicans by monocytes in the presence of IL-1a containing Th17 supernatants (FIG. 6d). Taken together, these findings strongly suggest that Th17 cells clear C. albicans infections not only via their production of IL-17, as previously thought (48), but also, to a significant extent, via the ability of a unique Th17-cell subset to produce IL-1a.

Cumulatively, the findings identifying GSDME pore formation in T cells as an exit strategy for proinflammatory IL-1a and the regulation of GSDME by the NLRP3 inflammasome-caspase-8-caspase-3 axis reveal a new mode of T-cell cytokine secretion that is associated with a proinflammatory subset of Th17 cells with antifungal TCR specificities. This provides new therapeutic targets for the modulation of human Th17 cells that are relevant for antifungal host defence and that might also participate in the pathogenesis of chronic inflammatory diseases.

### References as Cited

1. F. Sallusto, Heterogeneity of Human CD4(+) T Cells Against Microbes. Annu Rev Immunol 34, 317-334 (2016).
2. P. Zwicky, S. Unger, B. Becher, Targeting interleukin-17 in chronic inflammatory disease: A clinical perspective. J Exp Med 217, (2020).
3. B. Stockinger, S. Omenetti, The dichotomous nature of T helper 17 cells. Nat Rev Immunol 17, 535-544 (2017).
4. C. E. Zielinski, F. Mele, D. Aschenbrenner, D. Jarrossay, F. Ronchi, M. Gattorno, S. Monticelli, A. Lanzavecchia, F. Sallusto, Pathogen-induced human TH17 cells produce IFN-gamma or IL-10 and are regulated by IL-1beta. Nature 484, 514-518 (2012).
5. K. Ghoreschi, A. Laurence, X. P. Yang, C. M. Tato, M. J. McGeachy, J. E. Konkel, H. L. Ramos, L. Wei, T. S. Davidson, N. Bouladoux, J. R. Grainger, Q. Chen, Y. Kanno, W. T. Watford, H. W. Sun, G. Eberl, E. M. Shevach, Y. Belkaid, D. J. Cua, W. Chen, J. J. O'Shea, Generation of pathogenic T(H)17 cells in the absence of TGF-beta signalling. Nature 467, 967-971 (2010).
6. R. Noster, H. D. de Koning, E. Maier, M. Prelog, E. Lainka, C. E. Zielinski, Dysregulation of proinflammatory versus anti-inflammatory human TH17 cell functionalities in the autoinflammatory Schnitzler syndrome. J Allergy Clin Immunol 138, 1161-1169 e1166 (2016).
7. D. Aschenbrenner, M. Foglierini, D. Jarrossay, D. Hu, H. L. Weiner, V. K. Kuchroo, A. Lanzavecchia, S. Notarbartolo, F. Sallusto, An immunoregulatory and tissue-residency program modulated by c-MAF in human TH17 cells. Nat Immunol 19, 1126-1136 (2018).
8. J. Matthias, S. Heink, F. Picard, J. Zeitrag, A. Kolz, Y. Y. Chao, D. Soll, G. P. de Almeida, E. Glasmacher, I. D. Jacobsen, T. Riedel, A. Peters, S. Floess, J. Huehn, D. Baumjohann, M. Huber, T. Korn, C. E. Zielinski, Salt generates antiinflammatory Th17 cells but amplifies pathogenicity in proinflammatory cytokine microenvironments. J Clin Invest 130, 4587-4600 (2020).
9. G. Cavalli, S. Colafrancesco, G. Emmi, M. Imazio, G. Lopalco, M. C. Maggio, J. Sota, C. A. Dinarello, Interleukin 1alpha: a comprehensive review on the role of IL-1alpha in the pathogenesis and treatment of autoimmune and inflammatory diseases. Autoimmun Rev 20, 102763 (2021).
10. E. V. Acosta-Rodriguez, G. Napolitani, A. Lanzavecchia, F. Sallusto, Interleukins 1beta and 6 but not transforming growth factor-beta are essential for the differentiation of interleukin 17-producing human T helper cells. Nat Immunol 8, 942-949 (2007).
11. E. Latz, T. S. Xiao, A. Stutz, Activation and regulation of the inflammasomes. Nat Rev Immunol 13, 397-411 (2013).
12. J. Shi, Y. Zhao, K. Wang, X. Shi, Y. Wang, H. Huang, Y. Zhuang, T. Cai, F. Wang, F. Shao, Cleavage of GSDMD by inflammatory caspases determines pyroptotic cell death. Nature 526, 660-665 (2015).
13. X. Liu, Z. Zhang, J. Ruan, Y. Pan, V. G. Magupalli, H. Wu, J. Lieberman, Inflammasome-activated gasdermin D causes pyroptosis by forming membrane pores. Nature 535, 153-158 (2016).
14. O. Gross, A. S. Yazdi, C. J. Thomas, M. Masin, L. X. Heinz, G. Guarda, M. Quadroni, S. K. Drexler, J. Tschopp, Inflammasome activators induce interleukin-1 alpha secretion via distinct pathways with differential requirement for the protease function of caspase-1. Immunity 36, 388-400 (2012).
15. Ivanov, II, B. S. Mckenzie, L. Zhou, C. E. Tadokoro, A. Lepelley, J. J. Lafaille, D. J. Cua, D. R. Littman, The orphan nuclear receptor RORgammat directs the differentiation program of proinflammatory IL-17+ T helper cells. Cell 126, 1121-1133 (2006).
16. E. V. Acosta-Rodriguez, L. Rivino, J. Geginat, D. Jarrossay, M. Gattorno, A. Lanzavecchia, F. Sallusto, G. Napolitani, Surface phenotype and antigenic specificity of human interleukin 17-producing T helper memory cells. Nat Immunol 8, 639-646 (2007).
17. R. Noster, R. Riedel, M. F. Mashreghi, H. Radbruch, L. Harms, C. Haftmann, H. D. Chang, A. Radbruch, C. E. Zielinski, IL-17 and GM-CSF expression are antagonistically regulated by human T helper cells. Sci Transl Med 6, 241ra280 (2014).
18. S. Eyerich, C. E. Zielinski, Defining Th-cell subsets in a classical and tissue-specific manner: Examples from the skin. Eur J Immunol 44, 3475-3483 (2014).
19. M. Keller, A. Ruegg, S. Werner, H. D. Beer, Active caspase-1 is a regulator of unconventional protein secretion. Cell 132, 818-831 (2008).
20. M. Monteleone, J. L. Stow, K. Schroder, Mechanisms of unconventional secretion of IL-1 family cytokines. Cytokine 74, 213-218 (2015).
21. B. Mosley, D. L. Urdal, K. S. Prickett, A. Larsen, D. Cosman, P. J. Conlon, S. Gillis, S. K. Dower, The interleukin-1 receptor binds the human interleukin-1 alpha precursor but not the interleukin-1 beta precursor. J Biol Chem 262, 2941-2944 (1987).
22. A. Malik, T. D. Kanneganti, Function and regulation of IL-1alpha in inflammatory diseases and cancer. Immunol Rev 281, 124-137 (2018).
23. L. M. Carruth, S. Demczuk, S. B. Mizel, Involvement of a calpain-like protease in the processing of the murine interleukin 1 alpha precursor. J Biol Chem 266, 12162-12167 (1991).
24. Y. Kobayashi, K. Yamamoto, T. Saido, H. Kawasaki, J. J. Oppenheim, K. Matsushima, Identification of calcium-activated neutral protease as a processing enzyme of human interleukin 1 alpha. Proc Natl Acad Sci USA 87, 5548-5552 (1990).
25. I. S. Afonina, G. A. Tynan, S. E. Logue, S. P. Cullen, M. Bots, A. U. Luthi, E. P. Reeves, N. G. McElvaney, J. P. Medema, E. C. Lavelle, S. J. Martin, Granzyme B-dependent proteolysis acts as a switch to enhance the proinflammatory activity of IL-1alpha. Mol Cell 44, 265-278 (2011).
26. A. H. Chan, K. Schroder, Inflammasome signaling and regulation of interleukin-1 family cytokines. J Exp Med 217, (2020).
27. A. Fettelschoss, M. Kistowska, S. LeibundGut-Landmann, H. D. Beer, P. Johansen, G. Senti, E. Contassot, M. F. Bachmann, L. E. French, A. Oxenius, T. M. Kundig, Inflammasome activation and IL-1beta target IL-1alpha for secretion as opposed to surface expression. Proc Natl Acad Sci USA 108, 18055-18060 (2011).
28. B. S. Franklin, E. Latz, F. I. Schmidt, The intra-and extracellular functions of ASC specks. Immunol Rev 281, 74-87 (2018).
29. A. Nagar, R. A. DeMarco, J. A. Harton, Inflammasome and Caspase-1 Activity Characterization and Evaluation: An Imaging Flow Cytometer-Based Detection and Assessment of Inflammasome Specks and Caspase-1 Activation. J Immunol 202, 1003-1015 (2019).
30. E. A. Kurt-Jones, D. I. Beller, S. B. Mizel, E. R. Unanue, Identification of a membrane-associated interleukin 1 in macrophages. Proc Natl Acad Sci USA 82, 1204-1208 (1985).
31. P. Broz, P. Pelegrin, F. Shao, The gasdermins, a protein family executing cell death and inflammation. Nat Rev Immunol 20, 143-157 (2020).
32. C. Antonopoulos, H. M. Russo, C. El Sanadi, B. N. Martin, X. Li, W. J. Kaiser, E. S. Mocarski, G. R. Dubyak, Caspase-8 as an Effector and Regulator of NLRP3 Inflammasome Signaling. J Biol Chem 290, 20167-20184 (2015).
33 P. R. Vajjhala, A. Lu, D. L. Brown, S. W. Pang, V. Sagulenko, D. P. Sester, S. O. Cridland, J. M. Hill, K. Schroder, J. L. Stow, H. Wu, K. J. Stacey, The Inflammasome Adaptor ASC Induces Procaspase-8 Death Effector Domain Filaments. J Biol Chem 290, 29217-29230 (2015).
34 H. R. Stennicke, J. M. Jürgensmeier, H. Shin, Q. Deveraux, B. B. Wolf, X. Yang, Q. Zhou, H. M. Ellerby, L. M. Ellerby, D. Bredesen, D. R. Green, J. C. Reed, C. J. Froelich, G. S. Salvesen, Pro-caspase-3 is a major physiologic target of caspase-8. J Biol Chem 273, 27084-27090 (1998).
35. C. A. Dinarello, Interleukin-1 in the pathogenesis and treatment of inflammatory diseases. Blood 117, 3720-3732 (2011).
36. P. Miossec, T. Korn, V. K. Kuchroo, Interleukin-17 and type 17 helper T cells. N Engl J Med 361, 888-898 (2009).
37. G. Arbore, E. E. West, R. Spolski, A. A. B. Robertson, A. Klos, C. Rheinheimer, P. Dutow, T. M. Woodruff, Z. X. Yu, L. A. O'Neill, R. C. Coll, A. Sher, W. J. Leonard, J. Kohl, P. Monk, M. A. Cooper, M. Arno, B. Afzali, H. J. Lachmann, A. P. Cope, K. D. Mayer-Barber, C. Kemper, T helper 1 immunity requires complement-driven NLRP3 inflammasome activity in CD4(+) T cells. Science 352, aad1210 (2016).
38. B. N. Martin, C. Wang, C. J. Zhang, Z. Kang, M. F. Gulen, J. A. Zepp, J. Zhao, G. Bian, J. S. Do, B. Min, P. G. Pavicic, Jr., C. El-Sanadi, P. L. Fox, A. Akitsu, Y. Iwakura, A. Sarkar, M. D. Wewers, W. J. Kaiser, E. S. Mocarski, M. E. Rothenberg, A. G. Hise, G. R. Dubyak, R. M. Ransohoff, X. Li, T cell-intrinsic ASC critically promotes T(H)17-mediated experimental autoimmune encephalomyelitis. Nat Immunol 17, 583-592 (2016).
39. G. S. Lee, N. Subramanian, A. I. Kim, I. Aksentijevich, R. Goldbach-Mansky, D. B. Sacks, R. N. Germain, D. L. Kastner, J. J. Chae, The calcium-sensing receptor regulates the NLRP3 inflammasome through Ca2+ and cAMP. Nature 492, 123-127 (2012).
40. G. Doitsh, N. L. Galloway, X. Geng, Z. Yang, K. M. Monroe, O. Zepeda, P. W. Hunt, H. Hatano, S. Sowinski, I. Munoz-Arias, W. C. Greene, Cell death by pyroptosis drives CD4 T-cell depletion in HIV-1 infection. Nature 505, 509-514 (2014).
41. Y. Gao, K. Deason, A. Jain, R. A. Irizarry-Caro, I. Dozmorov, L. A. Coughlin, I. Rauch, B. M. Evers, A. Y. Koh, E. K. Wakeland, C. Pasare, Transcriptional profiling identifies caspase-1 as a T cell-intrinsic regulator of Th17 differentiation. J Exp Med 217, (2020).
42. S. Xia, Z. Zhang, V. G. Magupalli, J. L. Pablo, Y. Dong, S. M. Vora, L. Wang, T. M. Fu, M. P. Jacobson, A. Greka, J. Lieberman, J. Ruan, H. Wu, Gasdermin D pore structure reveals preferential release of mature interleukin-1. Nature 593, 607-611 (2021).
43. Z. Zhang, Y. Zhang, S. Xia, Q. Kong, S. Li, X. Liu, C. Junqueira, K. F. Meza-Sosa, T. M. Y. Mok, J. Ansara, S. Sengupta, Y. Yao, H. Wu, J. Lieberman, Gasdermin E suppresses tumour growth by activating anti-tumour immunity. Nature 579, 415-420 (2020).
44. Y. Wang, W. Gao, X. Shi, J. Ding, W. Liu, H. He, K. Wang, F. Shao, Chemotherapy drugs induce pyroptosis through caspase-3 cleavage of a gasdermin. Nature 547, 99-103 (2017).
45. K. A. Wiggins, A. J. Parry, L. D. Cassidy, M. Humphry, S. J. Webster, J. C. Goodall, M. Narita, M. C. H. Clarke, IL-1α cleavage by inflammatory caspases of the noncanonical inflammasome controls the senescence-associated secretory phenotype. Aging cell 18, e12946-e12946 (2019).
46. C. Helmstetter, M. Flossdorf, M. Peine, A. Kupz, J. Zhu, A. N. Hegazy, M. A. Duque-Correa, Q. Zhang, Y. Vainshtein, A. Radbruch, S. H. Kaufmann, W. E. Paul, T. Höfer, M. Löhning, Individual T helper cells have a quantitative cytokine memory. Immunity 42, 108-122 (2015).
47. S. Ruhl, K. Shkarina, B. Demarco, R. Heilig, J. C. Santos, P. Broz, ESCRT-dependent membrane repair negatively regulates pyroptosis downstream of GSDMD activation. Science 362, 956-960 (2018).
48. Puel, A. et al. Chronic mucocutaneous candidiasis in humans with inborn errors of interleukin-17 immunity. Science 332, 65-68 (2011).
49. Wawrzyniak. M. et al. A novel, dual cytokine-secretion assay for the purification of human Th22 cells that do not co-produce IL-17A. Allergy 71, 47-57 (2016).

1. A method for diagnosing an inflammatory disease in a human patient, comprising detecting IL-1α producing Th17 cells in a sample comprising T cells obtained from said human patient comprising detecting gasdermin E protein expression, wherein the presence of said IL-1α producing Th17 cells is indicative of an inflammatory disease in the human patient, wherein the IL-1α as produced by the Th17 cells is secreted.

2. The method according to claim 1, wherein the detection of the gasdermin E protein expression comprises detection of gasdermin E protein pore formation.

3. The method according to claim 1, further comprising detecting at least one marker selected from the group consisting of NLRP3 inflammasome formation, calpain activity, caspase-3 activity, and caspase-8 activity in said IL-1α producing Th17 cells.

4. The method according to claim 1, wherein the inflammatory disease is selected from the group consisting of an inflammation that is at least one of caused and exacerbated by IL-1α producing Th17 cells; an inflammation that is at least one of caused and related to danger signal IL-1α; autoinflammatory Schnitzler syndrome; autoinflammatory disorder adult-onset Still's disease (AOSD); systemic-onset juvenile idiopathic arthritis; syndrome of periodic fever with aphthous stomatitis; pharyngitis; cervical adenitis (PFAPA); Behçet disease; chronic recurrent multifocal osteomyelitis (CRMO); chronic obstructive pulmonary disorder (COPD); an infection; a fungal infection; an inflammation of the human patient's lung caused by smoking; and gout.

5. The method according to claim 1, further comprising the step of detecting a relative amount of the IL-1α producing Th17 cells per at least one of volume of the sample and per overall Th17 cell population in said sample, further comprising the step of comparing the relative amount of the IL-1α producing Th17 cells as detected to at least one of a control sample and an earlier sample taken from the same human patient.

6. The method according to claim 5, further comprising diagnosing an exacerbated state of the inflammatory disease if an increase of the relative amount of the IL-1α producing Th17 cells is detected and diagnosing a reduced state of the inflammatory disease if a decrease of the relative amount of the IL-1α producing Th17 cells is detected.

7. A method for identifying an inflammation modulating compound, comprising the steps of:
a) contacting at least one inflammation modulating candidate compound with the pore forming part of human gasdermin E protein (GSDME-N), and
b) detecting one of an inhibition and increase of at least one of assembly and pore formation of GSDME-N in the presence of said candidate compound, when compared to an absence of said candidate compound,
wherein the one of inhibition and increase of the at least one of assembly and pore formation of GSDME-N identifies an inflammation modulating compound, wherein the method is performed one of in vitro and in a recombinant cell comprising a human Th17 cell lacking the gasdermin E gene.

8. A method for identifying and using an inflammation modulating compound, comprising the steps of:
a) contacting at least one inflammation modulating candidate compound with a cell expressing human gasdermin E protein,
b) inducing gasdermin E expression in said cell, and
c) detecting one of an inhibition and increase of at least one of assembly and pore formation of GSDME in the presence of said candidate compound, when compared to the absence of said candidate compound,
wherein the one of inhibition and increase of the at least one of assembly and pore formation of GSDME identifies an inflammation modulating compound.

9. The method according to 7, wherein the modulation is inhibition of assembly and pore formation of GSDME and the compound is an anti-inflammatory compound.

10. The method according to claim 8, wherein inducing gasdermin E expression in said cell comprises inducing one or more of NLRP3 inflammasome formation, calpain activity, caspase-3 activity, and caspase-8 activity.

11. The method according to 8, wherein the inhibition of the at least one of the assembly and pore formation of GSDME in the presence of said candidate compound comprises at least one of an inhibition of the expression of gasdermin E in said cell, an inhibition of expression of caspase-3 in said cell, and a reduction of at least one of expression and secretion of IL-1α of said cell.

12. The method according to claim 8, wherein the cell is a human Th17 cell.

13. The method according to claim 7, wherein the candidate compound is selected from the group consisting of a chemical molecule, a molecule selected from a library of small organic molecules, a molecule selected from a combinatory library, a cell extract, a plant cell extract, a small molecular drug, a protein, a protein fragment, a molecule selected from a peptide library, an antibody, and an antibody fragment.

14. The method according to claim 8, wherein said contacting is one of in vivo, in vitro, in solution, and with a solid carrier to which the candidate compound molecule is one of bound and conjugated.

15. The method according to claim 8, further comprising combining said anti-inflammatory compound, together with a pharmaceutically acceptable carrier, to obtain a pharmaceutical composition.

16. The method according to claim 8, further comprising at least one preventing and treating inflammation in a subject, the at least one prevention and treatment comprising administering to said subject an effective amount of at least one of the anti-inflammatory compound and a pharmaceutical composition comprising the anti-inflammatory compound, wherein the inflammation is selected from the group consisting of an inflammation that is one of caused and exacerbated by IL-1α producing Th17 cells; inflammation that is at least one of caused and related to danger signal IL-1α; autoinflammatory Schnitzler syndrome; autoinflammatory disorder adult-onset Still's disease (AOSD); systemic-onset juvenile idiopathic arthritis; syndrome of periodic fever with aphthous stomatitis; pharyngitis; cervical adenitis (PFAPA); Behçet disease; chronic recurrent multifocal osteomyelitis (CRMO); chronic obstructive pulmonary disorder (COPD); infection; a fungal infection; inflammation of the lung caused by smoking; and gout.

17. (canceled).

18. The method according to claim 16, further comprising:
(a) detecting an amount of IL-1α producing Th17 cells in a biological sample obtained from said subject; and
(b) comparing the amount as detected in step (a) with at least one of an amount in an earlier sample taken from said subject and a control sample.

19. The method according to claim 18, wherein a decrease of the amount of the IL-1α producing Th17 cells when compared to the at least one of the earlier sample taken from said subject and the control sample is indicative of at least one of the success of, progress of and sensitivity of at least one of the inflammation treatment prevention in the subject.

20. The method according to claim 16, wherein the subject further receives at least one of a second additional inflammation modulating prophylaxis a second additional inflammation modulating therapy.

21. The method according to claim 7, further comprising at least one preventing and treating inflammation in a subject, the at least one prevention and treatment comprising administering to said subject an effective amount of at least the anti-inflammatory compound and a pharmaceutical composition comprising the anti-inflammatory compound, wherein the inflammation is selected from the group consisting of an inflammation that is one of caused [and exacerbated by IL-1α producing Th17 cells; inflammation that is at least one of caused and related to danger signal IL-1α; autoinflammatory Schnitzler syndrome; autoinflammatory disorder adult-onset Still's disease (AOSD);
systemic-onset juvenile idiopathic arthritis; syndrome of periodic fever with aphthous stomatitis; pharyngitis; cervical adenitis (PFAPA); Behçet disease;
chronic recurrent multifocal osteomyelitis (CRMO); chronic obstructive pulmonary disorder (COPD); infection; a fungal infection; inflammation of the lung caused by smoking; and gout.
